(19) United States
(12) Patent Application Publication
(10) Pub. No.: US 2011/0052631 A1
(43) Pub. Date: Mar. 3, 2011

(54) INDUCTION OF PROLIFERATION, EFFECTOR MOLECULE EXPRESSION, AND CYTOLYTIC CAPACITY OF HIV-SPECIFIC CD8+ T CELLS

(75) Inventors: Mark Connors, Bethesda, MD (US); Stephen A. Migueles, Washington, DC (US)

(73) Assignee: The United States of America, As Represented By The Secretary, Department of, Bethesda, MD (US)

(21) Appl. No.: 12/934,522

(22) PCT Filed: Mar. 25, 2009
(86) PCT No.: PCT/US09/01859

§ 371 Date: Sep. 24, 2010

Related U.S. Application Data
(60) Provisional application No. 61/070,849, filed on Mar. 26, 2008.
(60) Provisional application No. 61/199,126, filed on Nov. 12, 2008.

(51) Int. Cl. A61K 39/21; C12N 5/078; C12N 5/0783; A61P 37/04; A61P 31/18

(52) U.S. Cl. 424/208.1; 435/325; 435/375

(57) ABSTRACT

Provided is a method of activating an immune cell of a subject with Human Immunodeficiency Virus (HIV), comprising contacting the immune cell with a phorbol ester and a calcium ionophore. Also provided is a composition comprising immune cells of a subject diagnosed with HIV, wherein the immune cells are activated by contact with a phorbol ester and a calcium ionophore. Methods of using the disclosed compositions are also disclosed.

### CROSS REFERENCE TO RELATED PATENT APPLICATION

This application claims the benefit of U.S. Provisional Application No. 61/070,849, filed on Mar. 26, 2008 and U.S. Provisional Application No. 61/199,126, filed on Nov. 12, 2008, both of which are hereby incorporated by reference in its entirety.

### FIELD OF THE DISCLOSURE

This disclosure relates generally to the field of immunology. Specifically, the disclosure relates to the response by human immune cells to Human Immunodeficiency Virus (HIV).

### BACKGROUND

Encouragement for the ultimate development of effective vaccines and immunotherapies that would limit HIV replication can be drawn from patients who are naturally occurring examples of immune system-mediated control. These rare individuals contain HIV for many years to levels below 50 copies of HIV-1 RNA/ml plasma without antiretroviral therapy (ART) (Migueles et al., 2000). Although definitions and terms have varied, individuals within the instant cohort (referred to as long-term non-progressors (LTNP)) have been infected for a median of 17 years yet have remained clinically well with stable CD4+ T-cell counts and viral loads of <50 copies/ml. Certain HLA class I alleles, namely B*57 and, to a lesser extent, B*27, are consistently overrepresented in this and other LTNP cohorts (Flores-Villanueva et al., 2001; Kaslow et al., 1996; Migueles et al., 2000). Direct and indirect lines of evidence in humans and animal models suggest that virus-specific CD8+ T-cells mediate this control, although the mechanisms by which this occurs remain unknown (reviewed in (Migueles et al., 2004)).

Over the past 10 years, new tools have permitted an extensive characterization of the HIV-specific T-cell response of LTNP (reviewed in (Migueles et al., 2004))(Bailey et al., 2006; Emu et al., 2005; Migueles and Connors, 2001; Migueles et al., 2003; Migueles et al., 2000; Navis et al., 2007; Tilton et al., 2007). Although it has long been suspected that LTNP maintain better HIV-specific CD8+ T-cell function than progressors, a clear effector mechanism has not been found. The HIV-specific CD8+ T-cells of LTNP maintain greater frequencies of “polyfunctional” cells, named for their ability to degranulate and to produce several cytokines including IL-2 (Betts et al., 2006; Zimmerli et al., 2005). However, these cells make up an extremely small subset of the total HIV-specific CD8+ T-cell response and many LTNP demonstrate few or no such cells. In addition, the manner in which these cellular properties lead to improved immunologic control of HIV is unclear.

HIV-specific CD8+ T-cells of LTNP maintain greater proliferative capacity than those of progressors and upregulate perforin upon stimulation (Arrode et al., 2005; Horton et al., 2006; 2004; Migueles et al., 2002). Cytotoxicity has been explored in some prior work but has not thus far distinguished patients with immunologic control of HIV (Cao et al., 1996; Harrer et al., 1996; Klein et al., 1995; Pantaleo et al., 1995). However, these early studies lacked sensitive viral load measurements that would permit recruitment of homogeneous cohorts with clear immunologic control of HIV and compared responses with progressor patients with a global decline in immunity. Interpretation of these studies is further complicated by the use of assays such as bulk or limiting dilution chromium release that are neither highly quantitative nor highly reproducible in humans. More recently, the ability of CD8+ T-cells of LTNP to diminish HIV replication in humanized mice or HIV p24 protein in culture supernatants was used as a measure of suppression of HIV replication (Lopez Bernaldo de Quiros et al., 2000; Saez-Cirion et al., 2007). However, such assays do not measure the true frequency of targets or effectors, or the mechanism of killing. They are not sufficiently powerful to determine if the mechanism responsible for differences in function is mediated by precursor frequency, precursor proliferation, preferential target or effector cell death, Fas mediated killing, killing by non-CD8+ T-cells, or secretion of chemokines, TNF, or suppressor factors. For these reasons HW-specific cytotoxicity is not being measured in most laboratories in this field or in current vaccine trials.

Furthermore, the relationship of changes in lytic granule contents following stimulation and killing by virus-specific memory CD8+ T cells has remained poorly understood. It has been an accepted paradigm for some time that memory cells maintain full cytotoxic capacity (reviewed in (Seder and Ahmed, 2003; Trambas and Griffiths, 2003)). Barber et al. observed that lymphocytic choriomeningitis virus (LCMV)-specific memory cells in immune mice retained the ability to kill peptide pulsed targets in 1-4 hours suggesting that upregulation of effector molecules is not necessary for killing by memory cells (Barber et al., 2003). Based upon this model, re-expansion of memory cells does not involve qualitative changes in cytotoxic capacity but rather is primarily quantitative. More recently, other results have suggested that in vitro cytolytic capacity is related to the effector molecule content of memory LCMV-specific CD8+ T cells and not the ability to degranulate (Wolint et al., 2004). Increases in effector molecule content of lytic granules do occur over 3-6 days of stimulation in human EBV-, CMV-, and HIV-specific CD8+ T cells (Meng et al., 2006; Migueles et al., 2002; Sandberg et al., 2001). However, the relationship between changes in lytic granule content and killing has been largely unexplored in part due to the lack of reagents for staining of some lytic granule contents in mice and the need for assays that measure killing on a per-cell basis. Thus, the relationship between lytic granule contents and qualitative changes in killing capacity has not been established.

### SUMMARY OF THE DISCLOSURE

In accordance with the purpose(s) of this invention, as embodied and broadly described herein, this invention, in one aspect, relates to a composition comprising immune cells of a subject with HIV, wherein the immune cells are activated by contact with a phorbol ester and a calcium ionophore.

In another aspect, the invention relates to a method of activating an immune cell of a subject with HIV, comprising contacting the immune cell with a phorbol ester and a calcium ionophore, whereby contacting the cell with the phorbol ester and the calcium ionophore activates the cell.

In yet another aspect, the invention relates to a method of producing an immune response in a cell from a subject with HW directed against an HIV-infected cell, comprising contacting the immune cell with a phorbol ester and a calcium ionophore, whereby contacting the immune cell with the phorbol ester and the calcium ionophore produces an immune response in the cell directed against the HIV-infected cell.

In another aspect, the invention relates to a method of increasing production of an effector molecule in an immune cell of a subject with HIV, comprising contacting the immune cell with a phorbol ester and a calcium ionophore, whereby contacting the immune cell with the phorbol ester and the calcium ionophore increases production of the effector molecule in the cell.

In yet another aspect, the invention relates to a method of restoring to an immune cell of a subject with HIV the ability to proliferate, comprising contacting the immune cell with a phorbol ester and a calcium ionophore, whereby contacting the cell with the phorbol ester and the calcium ionophore restores to the immune cell the ability to proliferate.

In another aspect, the invention relates to a method of increasing the cytotoxicity of a CD8⁺ T-cell for HIV-infected CD4⁺ T-cells of a subject with progressive HIV infection, comprising contacting the CDC T-cell with a phorbol ester and a calcium ionophore, whereby contacting the CDC T-cell with the phorbol ester and the calcium ionophore increases the cytotoxicity of the CD8⁺ T-cell for the HIV-infected CD4^{÷} T-cells of the subject.

In another aspect, the invention relates to a method of producing an immune response to an HW-infected cell in a subject with progressive HIV infection, comprising administering to the subject a composition comprising immune cells of the subject, wherein the immune cells are activated by contact with a phorbol ester and a calcium ionophore in vitro.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIGS. 1A-1D show HIV-Specific CD8+ T-Cells Persist at Higher Frequencies in LTNP Compared with Treated Progressors at Equally Low Levels of HIV-1 RNA. (A) HW-1 RNA levels quantified to 1 copy/ml were compared between LTNP (n=27; black circles) and treated progressors who maintain <50 copies/ml (Rx<50, n=50; open circles). (B) Summary data of total IFN-gamma-producing HIV-specific CD8+ T-cell frequencies for individuals in (A). (C) Comparison of HW-1 RNA levels between LTNP (n=19) and Rx<50 (n=28) with detectable viremia (≧1 copy/ml). (D) Summary data of total IFN-gamma-producing HIV-specific CD8+ T-cell frequencies for individuals in (C). Horizontal lines indicate median values. Comparisons were made using the Wilcoxon two-sample test. Only significant P values are shown.

FIGS. 2A-2D show Flow Cytometry Based Cytotoxicity Assay Measures Granzyme B Activity in Peptide-Pulsed PBMC Targets.

(A) Representative marker expression following 6-day Gag peptide stimulation (+/−6-hour re-stimulation) is shown for an HLA B*57+ LTNP (top row) and progressor (bottom row). Quadrant values indicate the percentage of gated CD8+ T-cells. (B) GrB activity in PBMC targets pulsed with 3 B57-restricted Gag epitopes after adding day 0 (D#0, center column) or day 6 (D#6, right column) cells in a representative B*57 LTNP (top row) and progressor (bottom row). Values in upper right corner indicate percentages of targets with increased fluorescence due to cleavage of GrB substrate. Values below the latter ones indicate GrB activity after subtracting background values (left column for D#0 cells and not shown for D#6 cells). (C) Light scatter characteristics of gated targets from (B). (D), Measurement of day 0 (left column) and day 6 (right column) peptide-specific CD8+ T-cells using 3 B57 HIV tetramers complexed to the same peptides used in (B, C). Values indicate the percentage of gated CD8 T-cells.

FIGS. 3A-3D show HIV-Specific CD8+ T-Cells from LTNP Mediate Greater Lysis of Peptide-Pulsed Targets than Cells from Progressors.

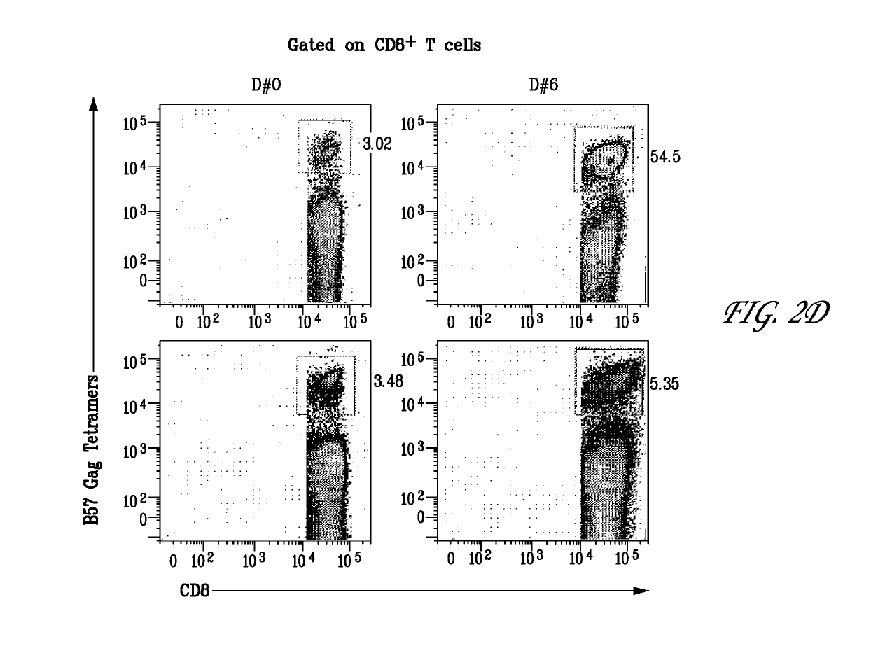

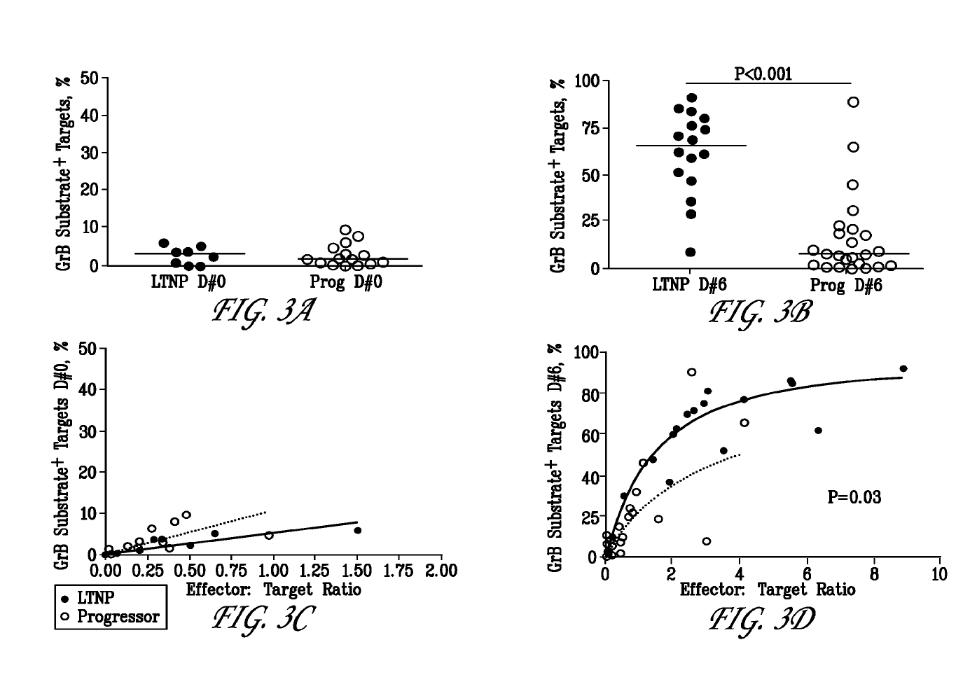

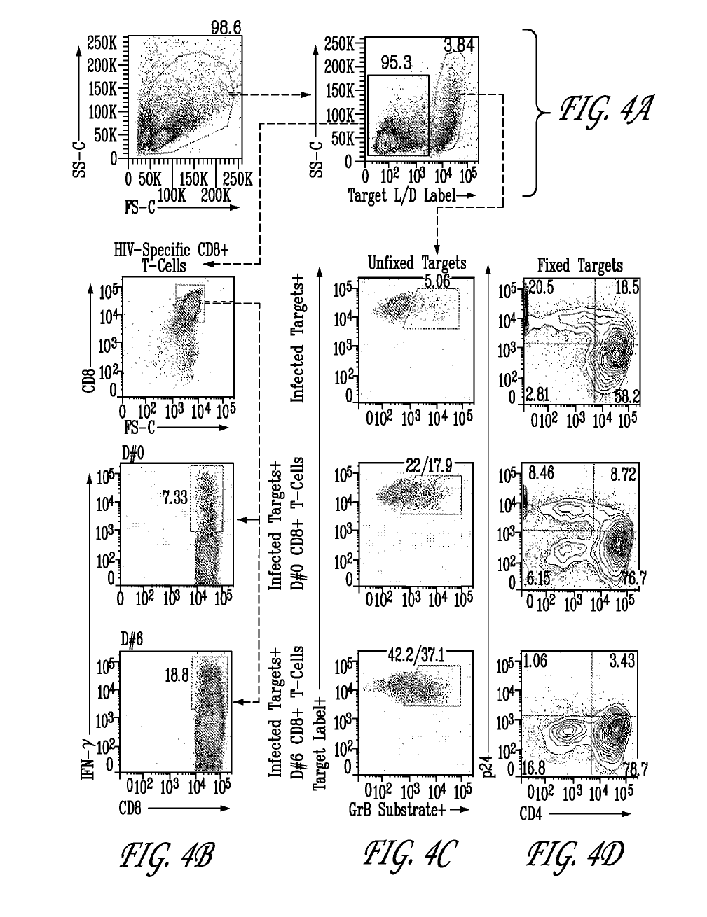

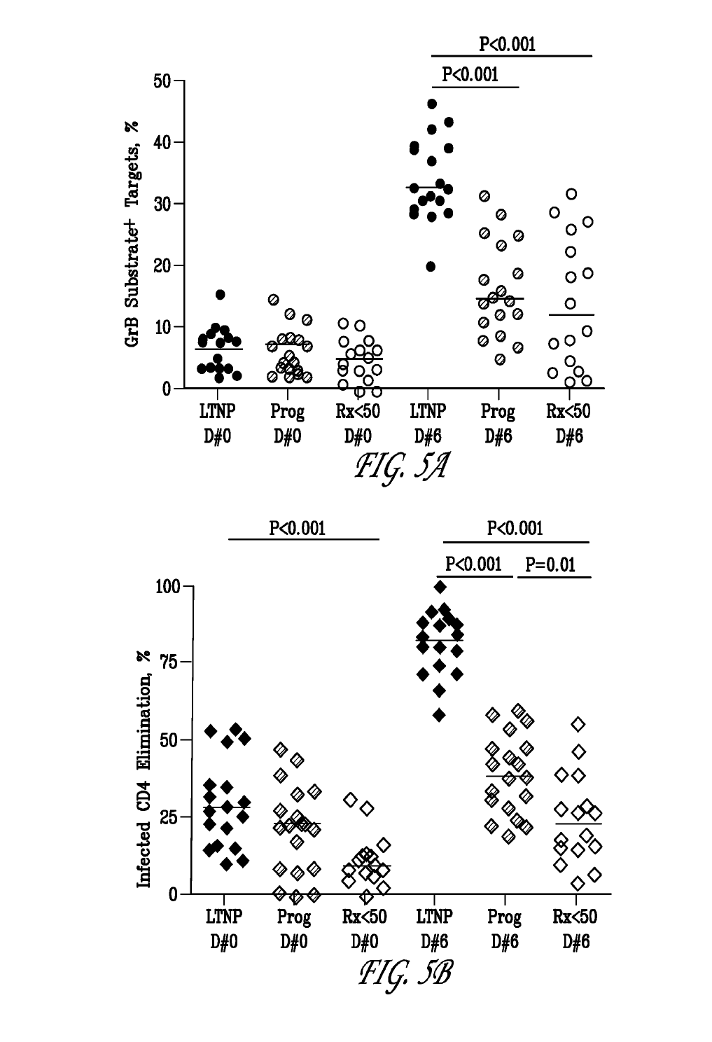

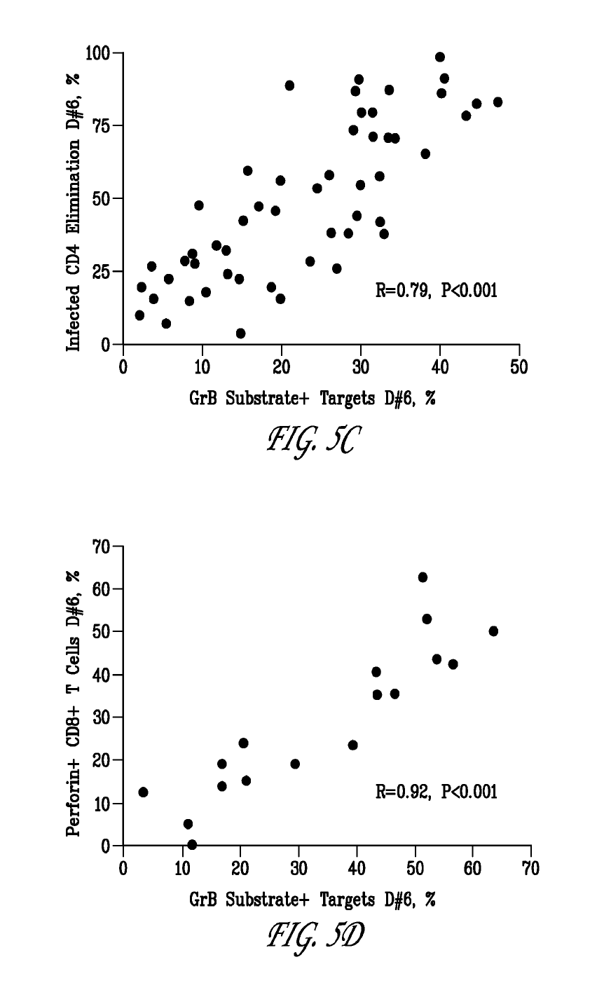

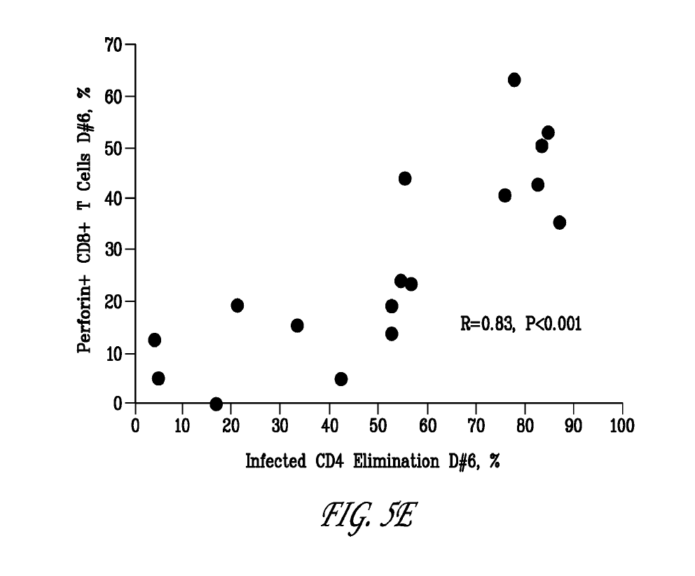

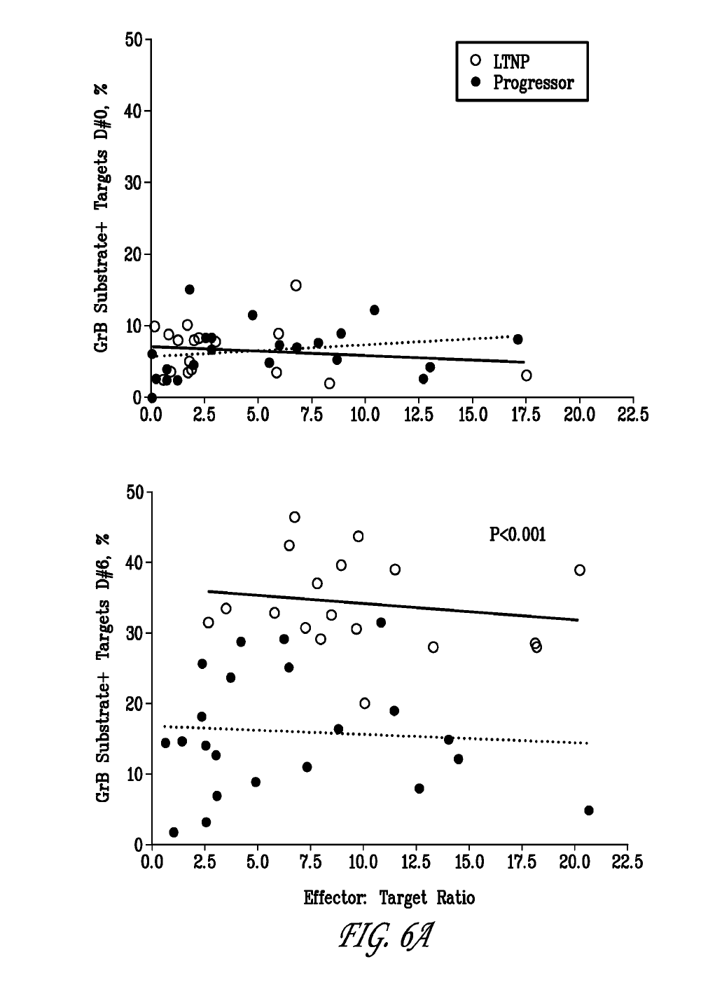

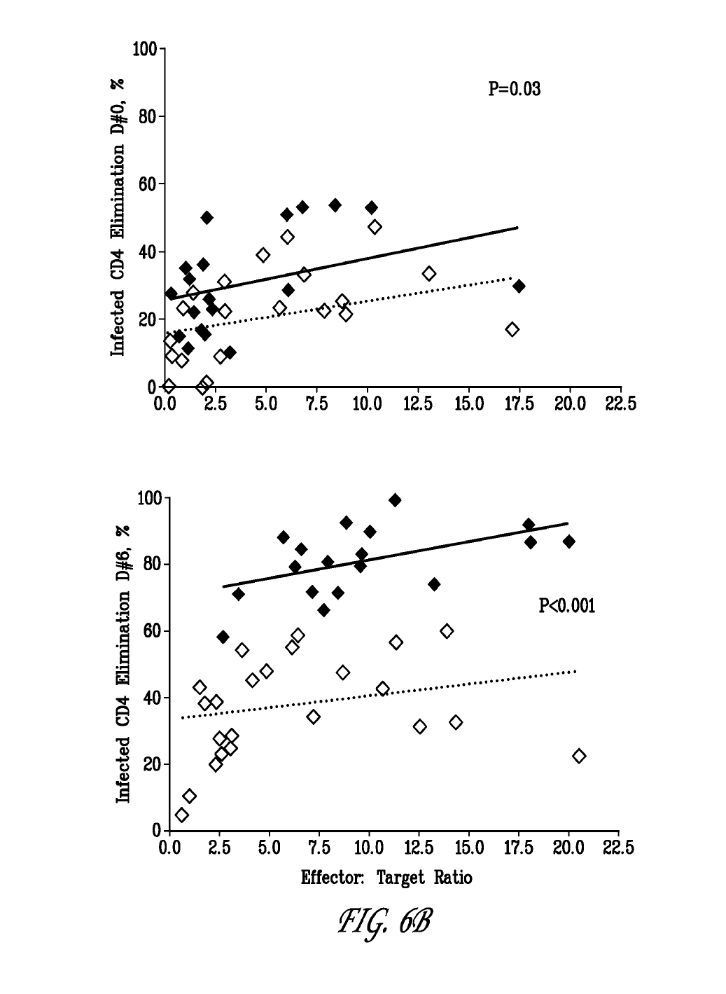

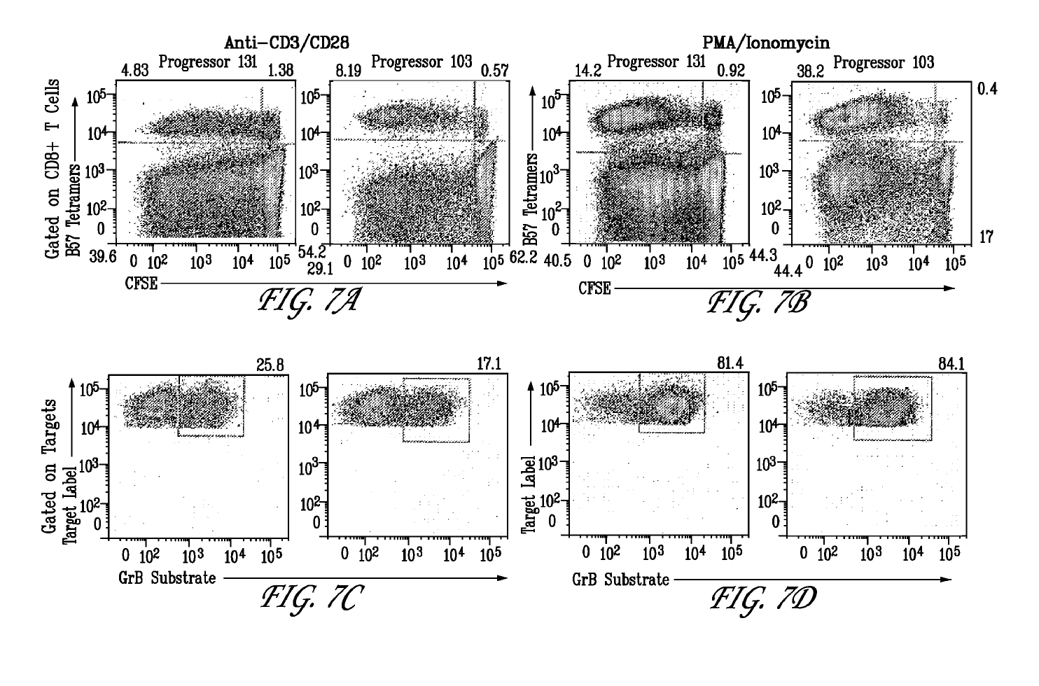

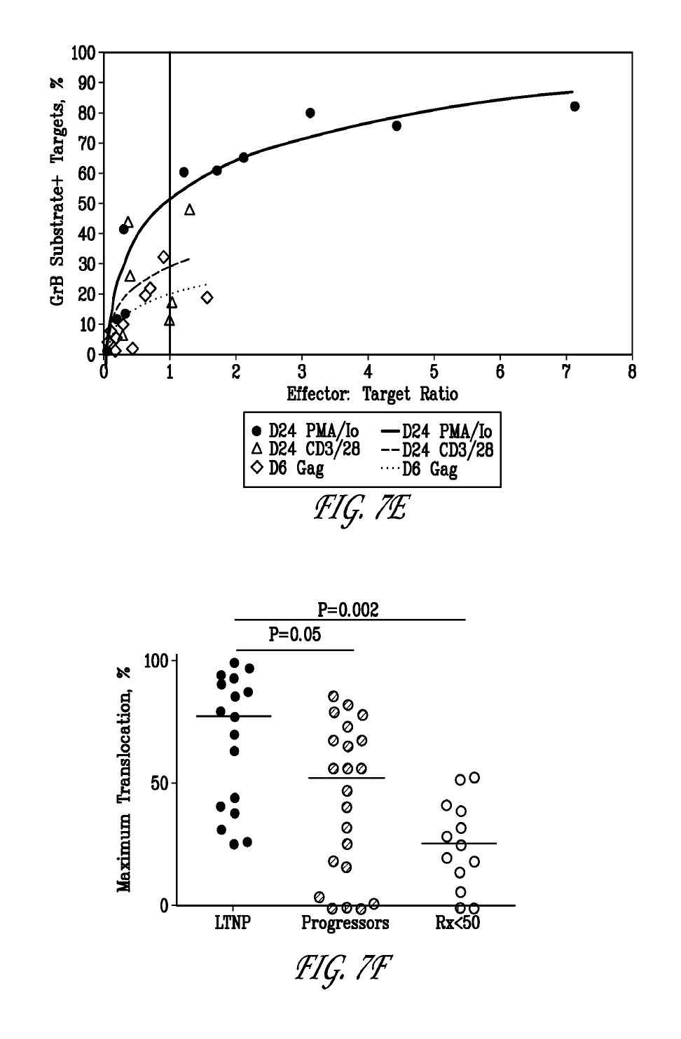

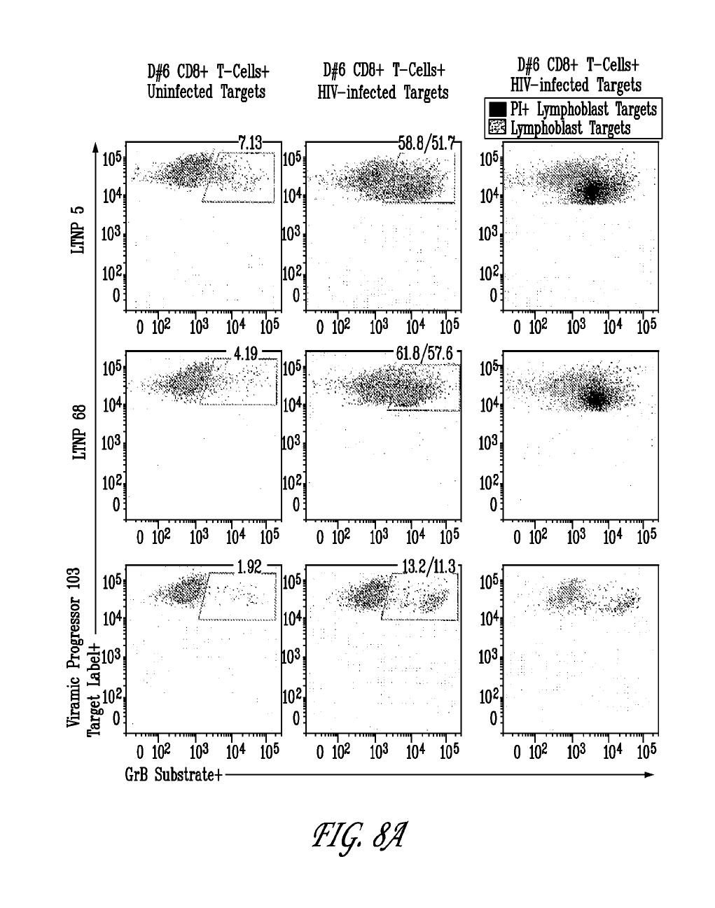

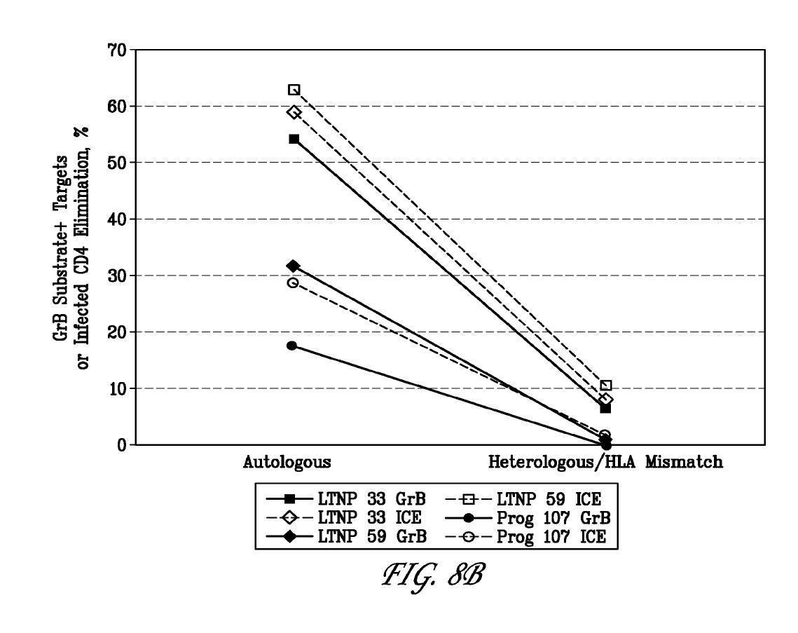

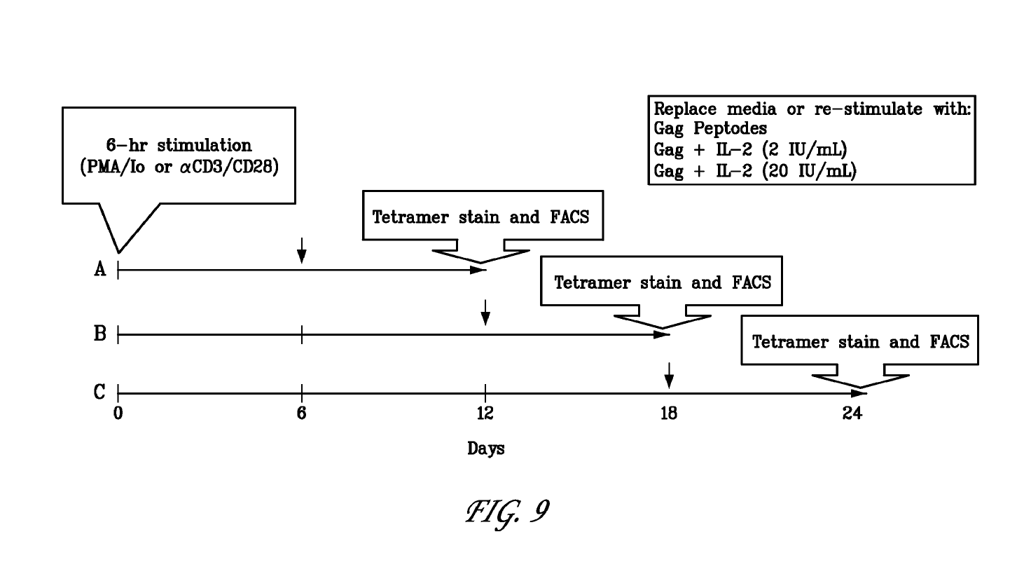

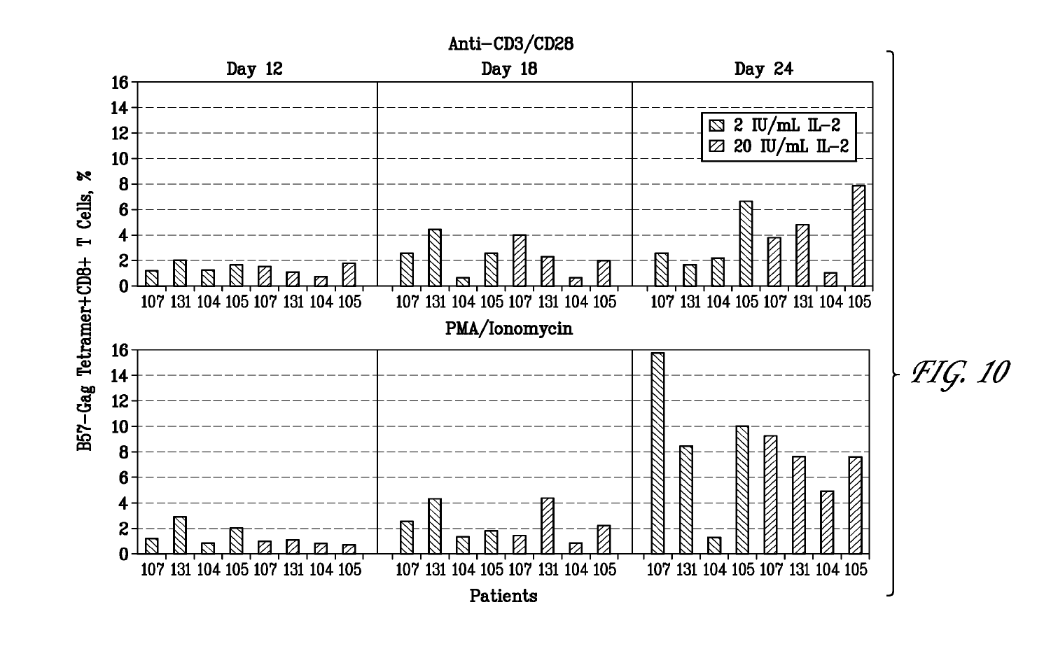

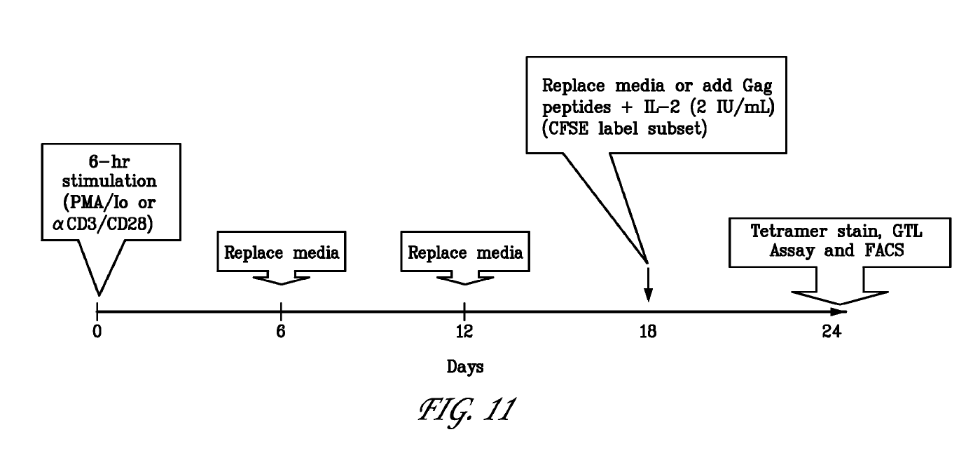

(A, B) Summary data of the total cytotoxic response (sum of the individual cytotoxic responses when more than one epitope was recognized) using day 0 cells of LTNP (black circles, n=8) and progressors (open circles, n=15, A) or day 6 cells of LTNP (n=16) and progressors (n=24, B). Horizontal lines indicate median values. Comparisons were made using the Wilcoxon two-sample test. Only significant P values are shown. (C, D) Data in (A) and (B) plotted against E:T ratios based on HIV-tetramer frequencies (FIG. 2 D). Curves represent trends for LTNP (black) and progressors (dotted). Analysis of covariance was used to quantify the logit of GrB activity in LTNP and progressors over the range of logged E:T ratios. Identical results were obtained if the analysis was limited to B27/57-restricted responses.

FIGS. 4A-4D show HIV-Specific CD8+ T-Cell Cytotoxicity Measured by Granzyme B Delivery or Infected CD4+ T-Cell Elimination.

(A) Plots showing gating scheme to identify 3 cell populations (right plot): CD8+ T-cell effectors (negative target LIVE/DEAD (L/D) label), CD4+ T-lymphoblast targets (positive target L/D label) and cells that have died prior to the incubation of CD8+ T-cells and CD4 + T-cell targets (high target L/D label, off scale and excluded from analysis). (B) Day 0 (D#0, middle panel) and Day 6 (D#6, bottom panel) HIV-specific CD8+ T-cells are measured as percentages of CD3+ CD8+ lymphocytes (top panel) expressing TN-gamma (see methods). (C) GrB activity in gated lymphoblast targets after adding no (top panel), day 0 (center panel) or day 6 (lower panel) CD8+ cells in a representative LTNP. In middle and bottom panels, the second values indicate percentages of targets with increased GrB activity above background. Background was determined by GrB activity in uninfected targets mixed with D#0 or D#6 cells, respectively. (D) Cells from (C) after fixation, permeabilization and staining for CD4 and intracellular p24 expression. Quadrant values indicate percentages of gated targets. Infected cell elimination (ICE) was calculated using p24+ targets (sum of upper quadrants) as described.

FIGS. 5A-5E show HIV-Specific CD8+ T-Cells from LTNP Mediate Greater Lysis of HIV-Infected CD4+ T-Cell Targets Compared with Progressors.

(A, B) Summary data of the total cytotoxic response using GrB activity (circles, A) or ICE (diamonds, B) in LTNP (black symbols, n=18), progressors (gray symbols, n=18 and 19, respectively) and Rx<50 (open symbols, n=16). Data are representative of three experiments. Comparisons were made using the Wilcoxon two-sample or signed rank tests. Horizontal lines indicate median values. Only significant P values are shown. Similar findings were obtained if the analysis was limited to B*27/57+ patients. (C) Using day 6 CD8+ T-cells, GrB target cell activity correlates directly with ICE in LTNP, viremic progressors and Rx<50 (n=52). (D, E) Using day 6 CD8+ T-cells, the perforin content of HIV-specific CD8+ T-cells was directly correlated with both GrB target cell activity (D) and ICE (E) in a subset of LTNP, viremic progressors and Rx<50 (n=18). Statistical analyses were performed using the Spearman correlation.

FIGS. 6A-6B show Day 6 HIV-Specific CD8+ T-Cells of LTNP Mediate Greater Cytotoxicity of HIV-Infected CD4+ T-Cell Targets on a Per-Cell Basis than Cells of Progressors.

(A, B) GrB activity (circles, A) or ICE (diamonds, B) using D#0 (top panels) or D#6 (bottom panels) cells plotted against true E:T ratios based on measurements of IFN-gamma-secreting cells (FIG. 4B) and p24-expressing targets (FIG. 4D, top panel). Curves represent trends for LTNP (black solid) and progressors (gray dotted). Analysis of covariance was used to quantify the difference in GrB activity and ICE in LTNP and progressors over the range of E:T ratios.

FIGS. 7A-7F show Phorbol Ester and Calcium Ionophore Treatment Produces Greater Increases in Cytotoxic Capacity of HIV Tetramer+ CD8+ T-Cells than Treatment with Anti-CD3/Anti-CD28 Antibodies.

(A-D) Following anti-CD3/anti-CD28 (A, C) or PMA/Io (B, D) treatment, PBMC were incubated for 18 days (fresh medium was replaced every 6 days) at 37° C. and then stimulated with Gag peptides and IL-2 (2 IU/ml) for 6 more days. Some cells, which had been CFSE-labelled on day 18, were stained with 3 B57- or 2 B27-HIV Gag tetramers and assessed for proliferation on day 24 (A, B). Quadrant values indicate percentages of gated CD8+ T-cells. GrB activity in peptide-pulsed targets was measured with non-CFSE-labelled, day 24 cells (C, D). Values indicate percentages of targets after subtracting background values (targets mixed with CD8+ T-cells without peptides). Results of 2 representative B*57+ progressors are shown. E, Summary data of GrB activity plotted against E:T ratios of day 6 cells incubated with Gag peptides (gray diamonds and dotted line) or 24 days following treatment with anti-CD3/anti-CD28 antibodies (open triangles and dashed line) or PMA/Io (black circles and solid line) in 3 B*27 and 7 B*57+ progressors. Data are representative of four experiments. Vertical line represents the median E:T ratio. Linear mixed and generalized estimating equations approaches were used for inference. (F) Summary data of NFAT nuclear translocation in HIV tetramer+ cells for LTNP (black circles, n=17), viremic progressors (gray circles, n=22) and Rx<50 (open circles, n=13). Data are representative of four experiments. Horizontal lines indicate median values. Comparisons were made using the Wilcoxon two-sample test. Only significant P values are shown.

FIGS. 8A-8B show Cytotoxic Responses to Autologous Primary HIV-Infected CD4+ T-Cell Targets, which Correlate with Propidium Iodide Uptake, are Mediated by HIV-specific CD8+ T-Cells in an HLA Class I-Restricted Fashion. (A) Following incubation with day 6 autologous CD8+ T-cells, GrB activity in uninfected (left column) or 11W-infected CD4+ T-cell lymphoblast targets (center and right columns) is shown in two representative LTNP (top and center rows) or a viremic progressor (bottom row). Propidium iodide (PI) uptake (black overlay) occurs in the same infected target cells exhibiting increased GrB activity (right column). (B) Target cell GrB activity (closed symbols and solid lines) and infected CD4 elimination (ICE, open symbols and dashed lines) were assessed in 2 LTNP (black symbols) and one progressor (gray symbols) using autologous or heterologous LTNP-derived HIV-infected CD4+ T-cell targets mismatched at all HLA class I loci. Cytotoxicity above background was abrogated in all 3 individuals supporting that these responses were mediated in an HILA class I-restricted fashion.

FIG. 9 shows a Time Course to Determine Optimal Stimulation Conditions to Expand HIV-Specific CD8+ T-Cells.

Cells were stimulated for 6 hours with PMA/Io or anti-CD3/anti-CD28 monoclonal antibodies, washed and plated. Cells were then rested for 6, 12 or 18 days (A-C, respectively) and then re-stimulated with Gag peptides and IL-2 (2 or 20 IU/ml) for another 6 days (indicated by large gray arrows) prior to tetramer staining and analysis. Top medium was replaced every 6 days with fresh medium. Tetramer staining was used to quantitate the frequencies of HIV-specific CD8+ T cells present at the end of the incubation.

FIG. 10 shows Phorbol Ester and Calcium Ionophore Treatment Produces Greater Expansion of HIV Tetramer+ CD8+ T-Cells than Treatment with Anti-CD3/Anti-CD28 Antibodies.

Percentage of CD8+ T-cells positive for 3 B57-HW Gag tetramers after 6-hour stimulation with anti-CD3/anti-CD28 antibodies (top row) or PMA/Io (bottom row) followed by 12 (left panel), 18 (middle panel) or 24 (right panel)-day total incubation in 4 B*57+ progressors. HIV-1 Gag peptides and 2 (black bars) or 20 (gray bars) IU/ml of IL-2 were added for the final 6 days. Data are representative of four experiments.

FIG. 11 shows Final Experimental Design to Rescue HIV-Specific CD8+ T-Cell Proliferation and Cytotoxicity.

Cells were stimulated for 6 hours with PMA/Io or anti-CD3/anti-CD28 monoclonal antibodies, washed and plated. Cells were then rested for 18 days with the top medium replaced every 6 days with fresh medium. On day 18, the cells were re-stimulated with Gag peptides and IL-2 (2 IU/ml) for another 6 days prior to analysis. On day 18, a subset of cells was also CFSE-labeled so that proliferation over the next 6 days in response to HIV antigens (Gag peptides) could be tracked. On day 24, proliferation (in the CFSE-labeled fraction) and killing capacity were measured for each patient under each set of conditions.

### DETAILED DESCRIPTION

The present disclosure may be understood more readily by reference to the following detailed description of preferred embodiments and the Examples included therein and to the Figures and their previous and following description.

Before the present compounds, compositions, articles, devices, and/or methods are disclosed and described, it is to be understood that this disclosure is not limited to specific synthetic methods, specific phorbol esters, or to particular calcium ionophores, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a phorbol ester” or “a calcium ionophore” includes mixtures of phorbol esters and/or calcium ionophores.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint:

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not. As used herein, “long-term non-progressors (LTNP)” are subjects with confirmed HIV infection who have remained clinically well with a negative history for opportunistic infections or malignancies, stable CD4⁺ T-cell counts, set point HIV-1 RNA levels below the lower limit of detection (<50 copies/ml in branched DNA-based VERSANT HIV-1 RNA assay version 3.0, Bayer Diagnostics, Tarrytown, N.Y.) and no ongoing antiretroviral or immunomodulatory therapy. As used herein, “progressors” are subjects with confirmed HIV infection who have a history of opportunistic diseases and/or a progressive decline in CD4⁺ T-cell counts and current or previously documented poor restriction of virus replication when not receiving antiretroviral therapy (HIV-1 RNA levels >5,000 copies/ml).

The percentages of the HIV-specific CD8+ T cells detectable in the peripheral blood that recognize HIV do not differ between LTNP and progressors in cross sectional studies where only single time points are measured. Similarities between patient groups in the frequencies of these cells led to a search for qualitative (rather than merely quantitative) features of the HIV-specific CD8+ T cell response that might differentiate LTNP from progressors.

The first major qualitative difference between these patient groups lay in the ability of HIV-specific CD8+ T cells from LTNP to proliferate, or divide, in vitro following an encounter with HIV-infected CD4+ T cells. An arrest or blockade in an early part of the cell cycle prevented the CD8+ T cells of progressors from proceeding all the way through and dividing. This preserved proliferative capacity in LTNP cells was linked to greater upregulation of perforin in the CD8+ T cells of LTNP. Perforin is a pore-forming protein that is contained as an inactive form within cytotoxic granules of CD8+ T cells along with the serine proteases, granzyme (Gr) A and B. These proteins are released only after the CD8+ T cell comes into contact with a target cell, which the CD8+ T cell specifically recognizes through its receptor to be expressing foreign (“non-self”) peptides on the surface of the target cell in the groove of a particular HLA protein. Once these proteins are released, perforin enables GrB to enter the target cell. GrB then induces a cascade of changes that eventually causes the target cell to die. Hence, perforin is the central mediator of the pathway predominantly used by CD8+ T cells to kill an infected cell.

Because CD8+ T cell proliferation is the important initial step in this process that leads to lytic granule loading and an increase in the numbers of cells that can efficiently kill an HIV-infected CD4+ T cell, what is needed in the art is a means for reversing the defect in CD8+ T cell proliferation observed in progressors, and a composition comprising immune cells activated by such method. Potent polyclonal stimulation with a phorbol ester and a calcium ionophore, a period of rest, and re-stimulation with HIV antigens in the presence of IL-2 in vitro can induce the cells of progressors to proceed through cell cycle and to undergo all of the listed downstream effects culminating in the elimination of HIV-infected CD4+ T cells and successful restriction of HIV replication.

For example, as shown in FIGS. 9 and 10, a 6-hour stimulation with a phorbol ester, for example, phorbol-12-myristate-13-acetate (PMA), and a calcium ionophore, for example, ionomycin (Io), (PMA/Io), followed by some washes to remove any residual PMA/Io and an 18-day (versus a 6- or 12-day) period of rest before re-stimulating these cells with IL-2 and HIV antigens for 6 more days (24-day total culture) induced the greatest proliferation of HIV-specific CD8+ T cells compared with other polyclonal stimuli (e.g., anti-CD3/anti-CD28). These cells exhibited significantly greater killing than cells from the same patients treated with other stimuli (FIG. 7). Furthermore, these increases in killing carried out by PMA/Io-treated CD8+ T cells in progressors were comparable to the results observed using LTNP CD8+ T cells not stimulated with PMA/Io.

Thus, provided is a composition comprising immune cells of a subject diagnosed with Human Immunodeficiency Virus (HIV), wherein the immune cells are activated by contact with a phorbol ester and a calcium ionophore. As used herein, a “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects. In one aspect, a human subject can be a “long term non-progressor (LTNP).” In another aspect, a human subject can be a “progressor.” An example of an immune cell is a CD8⁺ T-cell. In one aspect, the CD8⁺ T-cell is a memory cell known as an HIV-specific CD8⁺ T-cell. As used herein, an immune cell is “activated” when it is contacted in such a way as to cause an increase in cellular metabolism and RNA content resulting in upregulation of activation markers (e.g., CD69, CD25, CD38, HLA DR, PD-1, Ki67, etc.), expression of cytokines/chemokines (e.g., interferon-gamma, tumor necrosis factor (TNF)-alpha, macrophage inflammatory protein (MIP)-lbeta, interleukin (IL)-2, etc) or effector molecules, or cell division. Examples of effector molecules include, but are not limited to, perforin and serine proteases for example, granzyme A, granzyme B, and granzyme C. In one aspect, a disclosed immune cell can be contacted in vitro.

In one aspect, a disclosed phorbol ester can be phorbol-12-myristate-13-acetate (PMA). Examples of other phorbol esters include, but are not limited to, phorbol 12,13-dibutyrate and phorbol 12,13-diacetate. In another aspect, a disclosed calcium ionophore can be ionomycin (Io). Examples of other calcium ionophores include, but are not limited to, A23187 and Br-X-573A.

Further, provided is a method of activating an immune cell of a subject diagnosed with HIV, comprising contacting the immune cell with a phorbol ester and a calcium ionophore, whereby contacting the cell with the phorbol ester and the calcium ionophore activates the cell. In one aspect, the immune cell can be an HIV-specific CD8⁺ T-cell. Further, the CD8⁺ T-cell can be contacted in vitro with a phorbol ester, for example, PMA, and a calcium ionophore, for example, ionomycin.

For example, 10-fold serial dilutions of PMA (0.065, 0.65, 6.5, 65 and 650 nM) and ionomycin (0.02, 0.2, 2, 20, 200 μM) can be used to determine the optimal concentrations required to induce the greatest expansion of HIV-specific CD8⁺ T-cells. In one embodiment, incubating the cells for 6 hours at 37° Celsius in 6.5 nM of PMA and 0.2 μM ionomycin, washing them and then incubating for another 6 days can lead to the recovery of the highest frequencies of viable HIV-specific CD8⁺ T-cells. Alternatively, concentrations of PMA in the range of 5-50 nM and concentrations of ionomycin in the range of 0.2-2 μM can produce similar results. Thus, PMA can be used in concentrations in the range from about 0.065 to about 650 nM, including concentrations in between. Further, ionomycin can be used in concentrations in the range from about 0.02 to about 200 μM, including concentrations in between.

Because cell death overwhelmed cultures stimulated for periods exceeding 6 hours, the optimal duration of cell contact with PMA/Io is from about 5 to about 6 hours. Moreover, an 18-day period of rest (following a 6-hour stimulation) compared to a 6- or 12-day rest period and subsequent re-stimulation with HIV antigens and IL-2 can result in even higher frequencies of HIV-specific CD8⁺ T-cells (see FIG. 10). Therefore, a rest period in the range from about 16 to about 20 days, followed by stimulation for from about 5 to about 6 days with HIV antigens and IL-2 can yield comparably high frequencies of HIV-specific CD8⁺ T-cells.

Also provided is a method of producing an immune response in a cell from a subject with HW, comprising contacting the immune cell with a phorbol ester and a calcium ionophore, whereby contacting the immune cell with the phorbol ester and the calcium ionophore produces an immune response in the cell. In one aspect, the subject is a progressor. In one aspect, the immune cell can be an HW-specific CD8⁺ T-cell. Further, the CD8⁺ T-cell can be contacted in vitro with a phorbol ester, for example, PMA, and a calcium ionophore, for example, ionomycin.

Also provided is a method of increasing production of an effector molecule in an immune cell of a subject with HIV, comprising contacting the immune cell with a phorbol ester and a calcium ionophore, whereby contacting the immune cell with the phorbol ester and the calcium ionophore increases production of the effector molecule in the cell. In one aspect, the subject is a progressor. In one aspect, the immune cell can be an HIV-specific CD8⁺ T-cell. Further, the CD8⁺ T-cell can be contacted in vitro with a phorbol ester, for example, PMA, and a calcium ionophore, for example, ionomycin. Examples of effector molecules include, but are not limited to, granzyme A, granzyme B, granzyme C, and perforin.

Further provided is a method of restoring to an immune cell of a subject with HIV the ability to proliferate, comprising contacting the immune cell with a phorbol ester and a calcium ionophore, whereby contacting the cell with the phorbol ester and the calcium ionophore restores to the immune cell the ability to proliferate. In one aspect, the subject is a progressor. In one aspect, the immune cell can be an 11W-specific CD8⁺ T-cell. Further, the CD8⁺ T-cell can be contacted in vitro with a phorbol ester, for example, PMA, and a calcium ionophore, for example, ionomycin.

Also provided is a method of increasing the cytotoxicity of a CD8⁺ T-cell for CD4⁺ HIV-infected cells of a subject, comprising contacting the CD8⁺ T-cell with a phorbol ester and a calcium ionophore, whereby contacting the CD8⁺ T-cell with the phorbol ester and the calcium ionophore increases the cytotoxicity of the CD8⁺ T-cell for CD4⁺ HIV-infected cells of the subject. In one aspect, the subject is a progressor. In one aspect, the immune cell can be an 11W-specific CD8⁺ T-cell. Further, the CD8⁺ T-cell can be contacted in vitro with a phorbol ester, for example, PMA, and a calcium ionophore, for example, ionomycin.

Provided is a method of producing an immune response to HIV in a subject diagnosed with 11W, comprising administering to the subject a composition comprising immune cells of the subject, wherein the immune cells are activated by contact with a phorbol ester and a calcium ionophore in vitro. An exemplary phorbol ester is PMA; an exemplary calcium ionophore is Io. In one aspect, the subject is a progressor. The immune cell can be an HIV-specific CD8⁺ T-cell. After the CD8⁺ T-cell is contacted in vitro with a phorbol ester and a calcium ionophore, the disclosed composition can be washed to remove the phorbol ester and calcium ionophore prior to administering the composition to the subject. Methods of washing the disclosed composition are well-known in the art.

The frequency and cytotoxic function of HIV-specific CD8+ T-cells and their mechanism of killing autologous HW-infected CD4+ T-cells in patients with and without immunologic control of HIV is disclosed herein. Using a highly sensitive HIV RNA assay, it was observed that HIV-specific CD8+ T-cells of LTNP persist at higher frequencies in vivo than those of treated progressors with equally low antigen levels. In addition, assays were applied that permitted a highly quantitative examination of cytotoxicity, effector and target frequencies, delivery of functional granzyme B (GrB), and elimination of primary autologous HIV-infected CD4+ T-cells. HIV-specific CD8+ T-cells of LTNP exhibited extraordinary cytotoxic capacity on a per-cell basis against HIV-infected cells. CD8+ T-cells of progressors, although capable of activation and cytokine secretion, lysed HIV-infected cells poorly even at high true effector:target (E:T) ratios. Defects in killing were reversible using phorbol ester and calcium ionophore stimulation. These findings show that CD8+ T-cell loading and delivery of cytotoxic proteins to HW-infected CD4+ T-cells by CD8+ T-cells is an 11W-specific effector mechanism that clearly segregates with LTNP. HIV-specific CD8+ T cells capable of producing cytokines are present in progressors that are disrupted in the loading of lytic granules, which results in poor cytolytic capacity on a per-cell basis. Thus, lytic granule contents of memory cells are a critical determinant of cytotoxicity that must be induced for maximal per-cell killing capacity.

### Experimental

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their discovery. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Study Subjects

Subjects were recruited from the Clinical Research Center, National Institutes of Health (Bethesda, Md.) and signed National Institute of Allergy and Infectious Diseases Investigational Review Board-approved protocol informed consent documents. Human immunodeficiency virus (HIV) infection was documented by HW-½ immunoassay. LTNP criteria include: clinically healthy, negative history for opportunistic diseases, stable T-cell counts, set point HW-1 RNA levels <50 copies/ml (bDNA-based VERSANT HIV-1 RNA assay version 3.0, Bayer Diagnostics, Tarrytown, N.Y.) and no ongoing antiretroviral therapy (ART, Table 1). Progressors were divided into subgroups based on duration of infection, HIV-1 RNA set points and treatment status (Table 2). Untreated patients were either ART naïve or had been off ART for at least six months prior to leukapheresis. Treated patients received continuous ART and patients with <50 copies of HIV RNA/ml had been suppressed for a median of 5 years (range 2-11). Median durations of HIV infection for slow progressors, untreated progressors, treated progressors with detectable viremia and treated patients with <50 copies/ml were 21 (range 6-23), 14 (range 4-22), 18 (range 7-22) and 14 (range 3-22) years, respectively. Median CD4+ T-cell counts were 737 (range 557-1,040), 416 (range 238-790), 318 (range 224-463) and 599 (range 204-1,409) cells/ml, respectively. Median HIV-1 RNA levels were 11,820 (range 3,237-28,890), 82,483 (range 30,733-175,204), 6,364 (range 1,702-9,664) and <50 copies/ml, respectively. Peripheral blood mononuclear cells (PBMC) were obtained as described previously (Migueles et al., 2002). HLA class typing was performed by sequence-specific hybridization as described previously (Migueles et al., 2000).

### HIV-1 RNA Determination

In a subgroup of LTNP and treated patients with <50 copies/ml (Tables 1, 2), single copy assays were performed as described (Palmer et al., 2003). Briefly, patient plasma samples containing added avian retrovirus (to serve as an internal control) were centrifuged, extracted and subjected to reverse-transcription and real-time PCR to amplify a portion of HIV-1 gag. The single assay amplifies a 75-nucleotide sequence within gag; approximately 10% of sequences are not amplified, likely as a consequence of primer mismatch. Details of extraction amplification, internal controls, and performance characteristics have been previously described (Palmer et al., 2003). The limit of quantification is a function of the amount of plasma used for the assay; for these experiments using stored plasma, a limit of 1 copy HIV-1 RNA/ml plasma was employed.

### HIV_{SF162}-Infected Autologous CD4+ T-Cell Targets

CD4+ T-cells were positively selected from cryopreserved PBMC by magnetic automated cell sorting (AutoMACS, Miltenyi Biotec, Germany) and polyclonally stimulated prior to infection as previously described (Migueles et al., 2002). CD4+ lymphoblasts were infected as recently described (Sacha et al., 2007). Briefly, concentrated HIV_{SF162} was bound to ViroMag beads (OZ Biosciences, Marseille, France). CD4+ lymphoblasts were re-suspended in warmed medium containing IL-2 (Roche Diagnostics, Manheim, Germany) at 40 IU/ml and plated at 5×10⁵ cells/50 μl/well in 96-well flat-bottom tissue culture plates. Bead-labeled virus or medium was added to “infected” or “uninfected” control wells, respectively. The plates were centrifuged at 1600 RPM×2 minutes prior to 15-minute incubation on a magnet (OZ Biosciences). The volume was raised to 210 microliters with IL-2 medium and the plates were incubated at 37° C. for 40 hours prior to use as targets in intracellular cytokine detection assays, cytotoxicity assays, or to stimulate PBMC.

### Granzyme B Cytotoxicity Assay

In peptide-based assays, cryopreserved PBMC targets, which had been rested overnight, were re-suspended at 2×10⁶ cells/ml in 0.5% human AB (HAB; Gem Cell Gemini Bio-Products, Sacramento, Calif.) medium and incubated in medium or medium containing HLA class I-restricted optimal peptides (2.5-5 μM for each peptide, Multiple Peptide Systems, San Diego, Calif.; Table 3) for 1 hour at 37° C. During the final 15 minutes, pulsed and non-pulsed targets were stained with the TFL4 fluorescent label (GranToxiLux, OncoImmunin, Inc., Gaithersburg, Md.) (Packard et al., 2007) diluted 1000× at 37° C. Targets were washed with HAB medium and labeled with a LIVE/DEAD Fixable Violet Stain Kit (Molecular Probes/Invitrogen Detection Technologies, Eugene, Oreg.) per the manufacturer's instructions. Targets were washed and gently re-suspended in 0.5% HAB medium. PBMC, which had either been rested overnight (day 0) or stimulated with pooled HIV-1 Gag, Pol, Nef or Env Glade B consensus sequence overlapping 15 mer peptides (final concentration 2 μg/ml of each peptide, NIH AIDS Research and Reference Program) corresponding to the relevant optimal epitopes (day 6), were combined with targets at an E:T ratio of 25:1. Cells were centrifuged, re-suspended in 75 μl of GrB substrate (GranToxiLux, Oncolmmunin, Inc.) diluted 4×, plated in 96-well round bottom plates and incubated at 37° C. for 1 hour in the dark. An E:T ratio of 25:1 and co-incubation times of 1-6 hours provided optimal signal-to-noise ratios. After 1 hour, cells were washed, gently re-suspended in PBS/1% BSA, placed on ice and analyzed immediately by flow cytometry. Aliquots of day 0 and day 6 cells were stained with the appropriate HLA class I tetramers as described below in order to correct the E:T ratios for the true numbers of HW-specific CD8+ T-cells.

In assays using CD4+ lymphoblast targets infected with HIV_{SF162}, day 0 cells and day 6 cells (incubated with infected targets) were labeled with immuno-magnetic beads (CD8+ T-cell Isolation Kit II, Miltenyi Biotec) prior to negative selection of CD8+ T-cells by magnetic automated cell sorting. HIV_{SF162}-infected and uninfected targets were labeled with the LIVE/DEAD violet stain as described above. Cells were washed and gently re-suspended in 10% HAB medium, mixed with day 0 or day 6 enriched CD8+ T-cells at an E:T ratio of 25:1, centrifuged, re-suspended in diluted GrB substrate and incubated at 37° C. in the dark. After one hour, the cells were treated as described above. Following analysis by flow cytometry, cells (including targets not incubated with effectors) were transferred to new V-bottom tubes, centrifuged, fixed and permeabilized with Cytofix/Cytoperm (BD PharMingen, San Diego, Calif.) prior to staining with allophycocyanin (APC)-conjugated anti-CD4 (BD Biosciences, San Jose, Calif.) and anti-p24 antibodies (Kc57 RDI, Beckman Coulter, Inc., Fullerton, Calif.) to confirm infection and to measure elimination of p24-expressing cells. The E:T ratios were corrected as follows: the true effector numbers were adjusted based on the frequencies of IFN-gamma+ CD8+ T-cells detected in parallel replicates after a 6-hour incubation (see below) and the true target numbers were corrected based on the total percentages of HIV_{SF162}-infected (p24+) cells as determined by the sum of the percentages of the upper quadrants in plots containing only infected targets. Infected cell elimination (ICE) was calculated as follows: % p24 expression of infected targets minus % p24 expression of infected targets mixed with day 0 or day 6 cells divided by % p24 expression of infected targets×100.

### CD8+ T-Cell Stimulation Assays for Intracellular Protein Detection

PBMC, which had been stimulated for 6 days with peptides, were re-suspended in 10% HAB medium and incubated with pooled HIV-1 Gag peptides, costimulatory antibodies (anti-CD28 and anti-CD49d, 1 μg/ml; BD Biosciences) monensin (Golgi Stop, 0.7 μg/ml; BD Biosciences) and anti-CD107a (Pacific Blue, BD PharMingen) at 37° C. At 2 hours brefeldin-A (10 mg/ml; Sigma Aldrich, St. Louis, Mo.) was added to inhibit cytokine secretion. At 6 hours, the cells were washed and stained with surface antibodies or HLA class I tetramers prior to fixation, permeabilization and intracellular staining as described previously (Migueles et al., 2002).

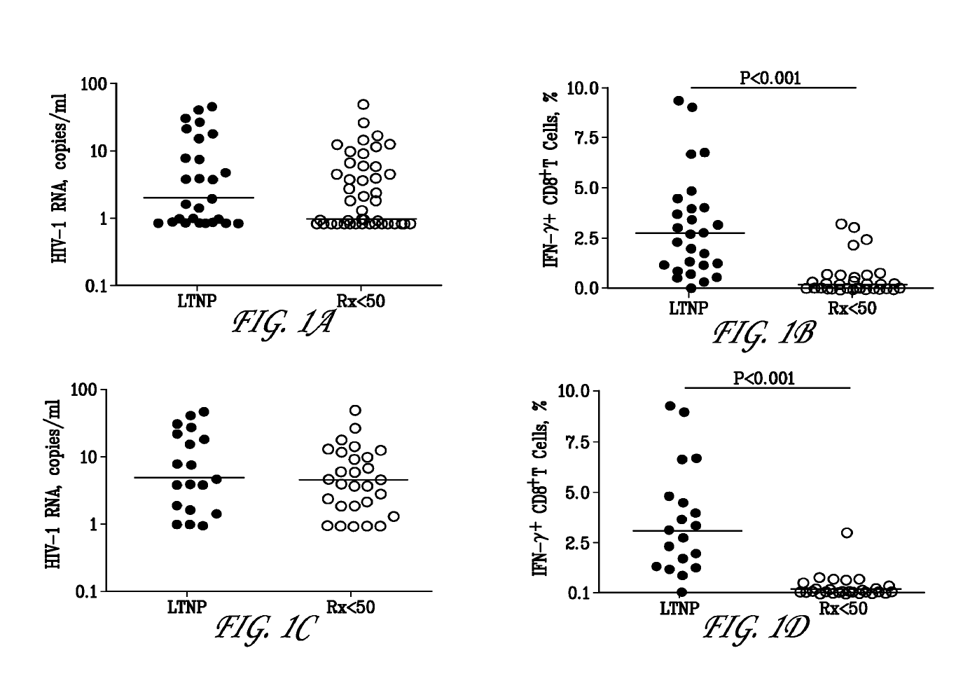

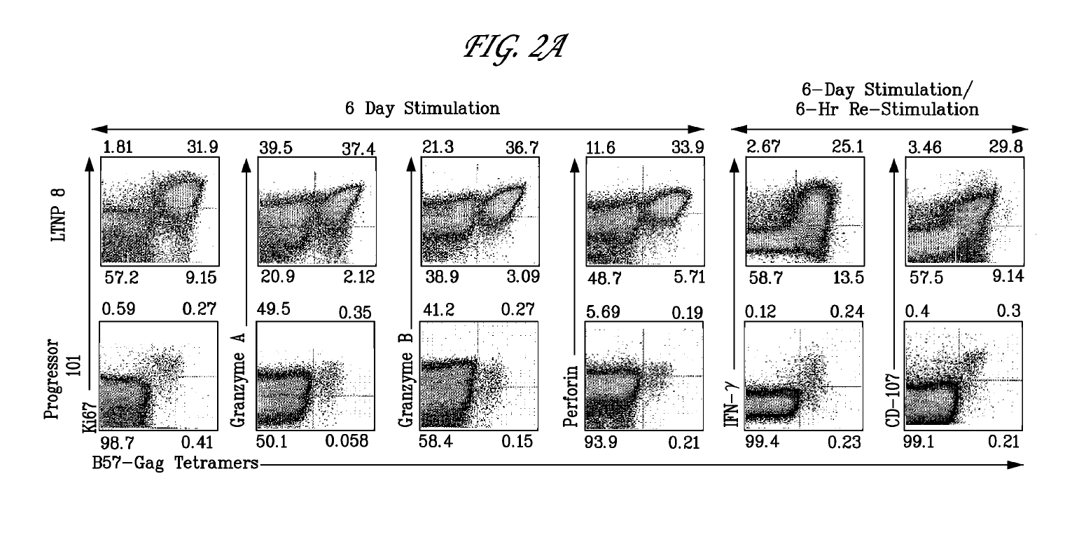

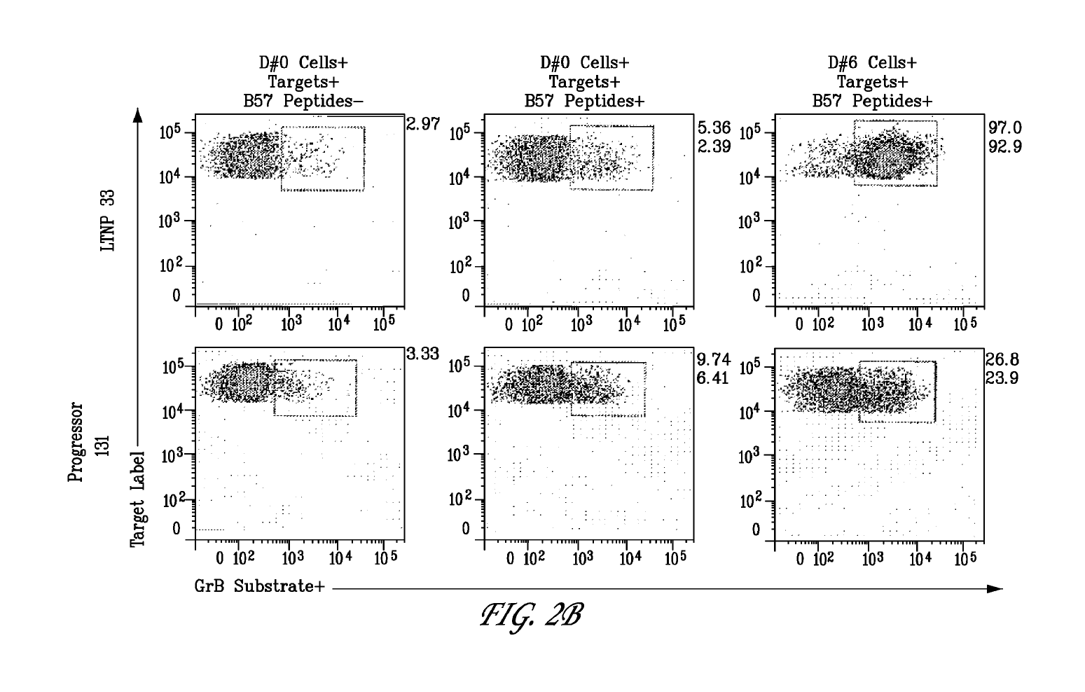

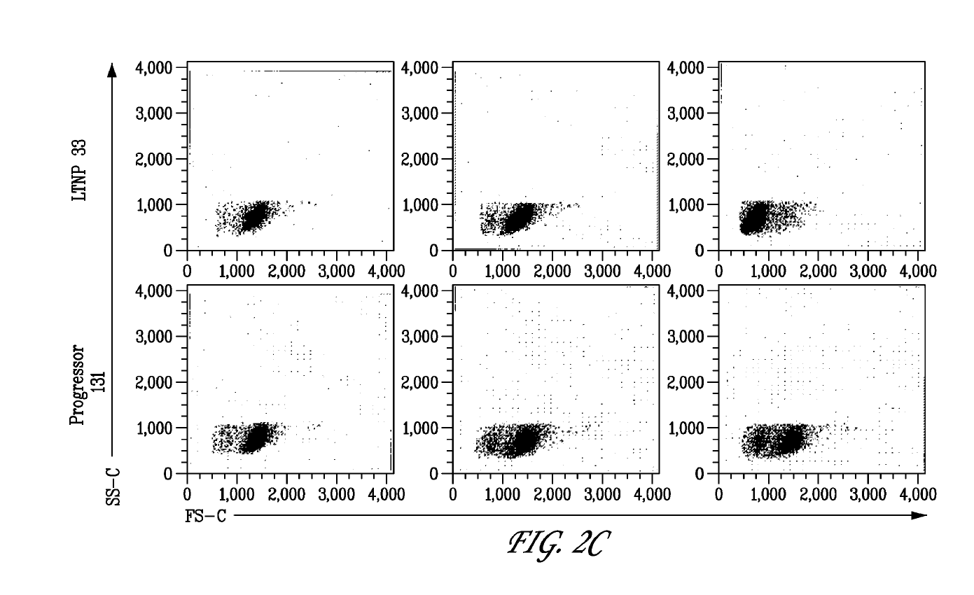

In experiments using CD4+ T-cell targets to measure the total frequency of virus-specific CD8+ T-cells, PBMC (rested overnight, FIG. 1) or negatively selected CD8+ T-cells (rested overnight or incubated with HIV_{SF162}-infected targets for 6 days, FIG. 4) were co-incubated with uninfected or HIV_{SF162}-infected autologous CD4+ T-cell targets at an E:T ratio of 1:1 as described previously (Migueles et al., 2002). At 6 hours, the cells were stained for surface markers prior to fixation, permeabilization and intracellular IFN-gamma staining as described previously (Migueles et al., 2000).

### CFSE Proliferation Assays

PBMC were labeled with 5,6-carboxyfluorescein diacetate, succinimidyl ester (CFSE; Molecular Probes, Eugene, Oreg.) as previously described (Migueles et al., 2002).

### Reversal Experiments

PBMC were re-suspended in 10% HAB medium to a concentration of 4×10⁶ cells/ml and polyclonally stimulated with phorbol-12-myristate-13-acetate (PMA, 6.5 nM; Calbiochem, Darmstadt, Germany) and ionomycin (Io, 0.2 μM; Sigma Aldrich) or anti-CD3 (Orthoclone OKT3, 1 μg/ml; Ortho Biotech, Bridgewater, N.J.) and anti-CD28 (1 μg/ml) antibodies at 37° C. At 6 hours, cells were incubated with DNAse I (10 U/ml; Invitrogen, Carlsbad, CA) at 37° C., washed, re-suspended in 10% HAB medium without (in the case of PMA/Io stimulated) or with anti-CD3/anti-CD28 antibodies and plated in 96-well, 1 ml deep-well culture plates (PGC Scientifics, Frederick, Md.) for 6, 12 or 18 days at 37° C. Unstimulated PBMC were also plated as controls. Medium was replaced every 6 days. At the conclusion of the incubation period, pooled HIV-1 Gag peptides with or without IL-2 (2 or 20 IU/ml) were added to the wells for 6 more days (12, 18 or 24 days total, respectively) at 37° C. prior to tetramer staining. Since a 24-day stimulation (18-day rest period followed by 6-day peptide re-stimulation) provided the highest frequencies of HIV tetramer+ CD8+ T-cells, PBMC were treated under these conditions prior to use in cytotoxicity assays. Some wells from each of the conditions were labeled with CFSE and analyzed for proliferation as described previously (Migueles et al., 2002).

### HLA Class I Tetramers

Seventeen HLA class I tetramers conjugated to either phycoerythrin (PE) or APC (Beckman Coulter, Inc.) were used to label epitope-specific CD8+ T-cells as previously described (Table 3) (Migueles et al., 2002).

### Flow Cytometry

Multiparameter flow cytometry was performed according to standard protocols. Surface and/or intracellular staining was done using the following directly conjugated antibodies obtained from BD Biosciences: fluorescein isothiocyanate (FITC)-conjugated anti-CD3; PE-conjugated anti-CD8; peridinine chlorophyll protein (PerCP)-conjugated anti-CD3; APC-conjugated anti-IFN-gamma; Pacific Blue-conjugated anti-PD-1 and anti-Granzyme A (GrA); PE Cy7-conjugated anti-perforin; Alexa 700-conjugated anti-Granzyme B (GrB); and APC Cy7-conjugated anti-Ki67. PE Alexa 700-conjugated anti-CD127 was purchased from Beckman Coulter. All staining was performed at 4° C. for 30 minutes. Flow cytometry profiles were gated on CD3+ CD8+ lymphocytes and 50,000-2×106 events were collected. In cytotoxicity experiments, gates were drawn on labeled PBMC or CD4+ T-cell targets and 5,000-8,000 events were collected. Samples were analyzed on a FACSAria multi-laser cytometer (Becton-Dickinson) with FACSDiva software. Color compensations were performed using single-stained samples for each of the fluorochromes used. Data were analyzed using FlowJo software (TreeStar, San Carlos, Calif.).

### Measurement of NFAT Translocation

PBMC were stimulated in 96 well deep plates at 2×10⁶ cells per well in a total volume of 500 μl/well. Cells were incubated in medium alone, or medium containing peptides (final concentration 2 μg/ml each) or PMA/Io (final concentration 400 nm each). Plates were incubated at 37° C. for 30 minutes then transferred to V-bottom tubes and chilled on ice for 10 minutes. Cells were then centrifuged and stained with anti-CD8 PE-Alexa 610 (Invitrogen) and the appropriate PE-labeled tetramer for 30 minutes at 4° C. Tetramers and corresponding peptides were chosen based upon the individual patient's HLA type as described above. Cells were then washed, fixed with 4% PFA, and permeabilized with 500 μl of a 1:1 mix of Phosflow buffers II and III (Becton-Dickinson) according to the manufacturer's protocol. The cells were then stained with Alexa 488-labeled anti-NFAT antibody (Becton-Dickinson), washed and resuspended in 120 μl of PBS/BSA containing 5 μM of DRAQ-5 nuclear stain (Alexis, Lausen, Switzerland).

Cell images were collected using an Image Stream 100 (Amnis, Seattle, Wash.) and analysis was performed similarly to a recently described technique (George et al., 2006). Briefly, the nuclear region of interest, or ‘nuclear mask,’ was determined based upon the contour of the DRAQ-5 image. The ‘cytoplasmic mask’ was created by subtracting the DRAQ-5 contour mask from the NFAT image contour mask. The ratio of the NFAT integral in the nuclear mask to the NFAT integral in the cytoplasmic mask was then used to create the similarity score. The percent of tetramer+ cells that translocated based upon similarity score was measured on 140,000 images per condition and expressed as % M, % P, and % PMA/Io (cells incubated in medium alone, with peptide, or with PMA/Io, respectively). The percent of maximum translocation was calculated as follows: (% P-% M)/(% PMA/Io-% M)×100=percent of maximum translocation.

### Statistical Analysis

The Wilcoxon signed rank test was used to compare paired data. Independent groups were compared by the Wilcoxon two-sample test. Correlation was determined by the Spearman rank method. The Bonferroni method was used to adjust p values for multiple testing. Analysis of covariance with appropriately transformed variables was used to quantify the difference in GrB activity and ICE of peptide-pulsed and HIV-infected CD4+ T-cell targets in LTNP and progressors over the range of E:T ratios. Linear mixed models and generalized estimating equations were used for analysis of the PMA/Io or anti-CD3/anti-CD28 reversal experiments.

### Results

HIV-Specific CD8+ T-Cell Frequencies are Higher in LTNP than in Treated Progressors Despite Similar Levels of HIV RNA

The relationship between frequency of HIV-specific CD8+ T cells and levels of viral antigen was examined. The frequency of HIV-spedific CD8+ T cellsin the peripheral blood of LTNP is no different from that of untreated progressors (Betts et al., 2001; Gea-Banacloche et al., 2000; Migueles et al., 2002). In contrast, progressors with HIV RNA levels suppressed to <50 copies/ml plasma by ART (Rx<50) have considerably lower HIV-specific CD8+ T-cell frequencies in the peripheral blood than LTNP or untreated progressors (Casazza et al., 2001; Gray et al., 1999; Migueles et al., 2002; Ogg et al., 1999a). It has been presumed that these differences between LTNP and Rx<50 are due to greater virus replication in LTNP below the detection threshold in standard assays. The relationship between viral replication and CD8+ T-cell frequency in these patient populations was examined (Tables 1 and 2) using a newer assay with a lower detection limit of 1 copy/ml (Palmer et al., 2003). Median HIV-1 RNA levels of Rx<50 were not significantly different from those of LTNP (1 versus 2 copies/ml, respectively, P=0.3, FIG. 1A). The median frequency of HIV-specific CD8+ T-cells producing interferon (IFN)-gamma in response to autologous HIV_{sF162}-infected CD4+ T-cells was 20-fold lower in Rx<50 than in LTNP (0.14% versus 2.8%, respectively, P<0.001; FIG. 1B). HW RNA was not detected in this assay in some LTNP or Rx<50. However, even when the analysis was limited to 19 LTNP and 28 ART recipients with plasma viremia greater than or equal to 1 copy/ml, the median frequency of HIV-specific CD8+ T-cells was still significantly lower in Rx<50 than in LTNP (0.13% versus 3.24%, respectively, P<0.001) despite similar plasma viral RNA levels (medians 5 versus 4.7 copies/ml plasma, respectively, P>0.5, FIGS. 1C and D). These data suggest that the HIV-specific CD8+ T-cells of LTNP persist at significantly higher frequencies than those in progressors with equally low antigen levels in vivo.

HIV-Specific CD8+ T-Cells from LTNP Mediate Greater Cytotoxicity of Peptide-Pulsed Targets than Cells from Progressors

Expression of effector proteins by HIV-specific CD8+ T-cells in LTNP and untreated progressors was compared (FIG. 2A). In unstimulated PBMC, differences in perforin or granzyme B content of HIV-specific cells between patient groups have not been detected (Appay et al., 2002; Migueles et al., 2002; Sandberg et al., 2001; Zhang et al., 2003). However, upon stimulation, the HIV-specific CD8+ T-cells of LTNP proliferate and upregulate perforin, with significant increases observed by day 3 and peak values measured by day 6. In the present study, analyses of effector protein expression were extended to include measurements of Ki67, granzymes A and B, and IFN-gamma. In addition, the ability to transport CD107a (lysosome-associated membrane protein-1) to the cell surface was used as a marker of degranulation capacity (Betts et al., 2003). Significantly higher frequencies of HIV Gag-tetramer+ CD8+ T-cells were detected in 8 LTNP compared with 8 progressors following a 6-day stimulation (medians 33.7% (17.4-53.3%) versus 3.77% (0.4-5.76%), respectively, P<0.001; FIG. 2A). Only the percent expression of granzyme B (GrB) and perforin (medians 87.85% (68.9-98.4%) versus 61.5% (40.4-85.3%), P<0.01; and 85.6% (69.7-96.6%) versus 46.4% (27.7-75.4%), P=0.004, respectively) and the MFI of perforin (medians 3,565.5 (1,639-6,348) versus 1,268 (303-2,728), P=0.01) were significantly higher in the tetramer+ cells of LTNP compared with those of progressors. Furthermore, GrB and perforin expression were very strongly correlated (R=0.87, P<0.001). Differences in the ability of HW-specific CD8+ T-cells to degranulate were not observed between LTNP and progressors (P>0.5). These findings support functional differences between the CD8+ T-cells of LTNP and progressors are not in the ability to degranulate but rather in the cytotoxic granule content (Meng et al., 2006; Migueles et al., 2002; Wolint et al., 2004).

Whether differences in cytotoxic granule content of HIV-specific CD8+ T-cells translated into differences in granule exocytosis-mediated cytotoxicity was next explored. Traditional assays of cytotoxicity are unable to differentiate whether differences in killing are due to differences in CD8+ T-cell proliferation. They are also unable to discern the mechanism of target cell killing. To determine if the increased GrB and perforin expression in HIV-specific CD8+ T-cells of LTNP was associated with increased granule exocytosis mediated HIV-specific cytotoxicity on a per-cell basis, a flow cytometry-based cytotoxicity assay was adapted that measures GrB-mediated intracellular cleavage of a cell permeable fluorogenic substrate in live targets (Packard et al., 2007). Using this technique, only functional GrB that had been delivered to the target cell, not inactive GrB stored within cytotoxic granules, is measured. FIG. 2B shows representative plots for a B*57+ LTNP (top row) and a B*57+ viremic progressor (bottom row). Cytotoxicity mediated by PBMC that were either rested overnight (“day 0” cells, left and middle columns) or incubated with Gag peptides for 6 days (“day 6” cells, right column) was assessed. Target PBMC were unpulsed (left column) or pulsed with immunodominant HLA B27/B57-restricted Gag optimal epitope peptides (middle and right columns): GrWmediated substrate cleavage was associated with characteristics of early death by light scatter as shown by the increased numbers of low forward scatter events in the samples with greater GrB activity (FIG. 2 C)(Packard et al., 2007). Similar changes in light scatter and GrB substrate cleavage were previously associated with apoptosis and death of target cells based upon annexin-V staining, morphologic changes such as membrane blebbing, and cell death based upon staining with propidium iodide (PI) or 7-amino-actinomycin D (Packard et al., 2007). To precisely quantitate the actual numbers of antigen-specific CD8+ T-cells present in the cultures, aliquots of day 0 (left column) and day 6 cells (right column) were stained with the appropriate HLA class I tetramers (FIG. 2D). Since the responses of progressors are broader than those of LTNP (Migueles et al., 2000), progressors were further screened with tetramers containing non-B27/57-restricted subdominant epitopes (Table 3). Responses were expressed as a sum of the individual cytotoxic responses when more than one epitope was recognized.

The total cytotoxic response of day 0 cells was low and not significantly different between LTNP and progressors (medians 3.09% versus 1.86%, respectively, P>0.5, FIG. 3A). However, day 6 cells of LTNP mediated dramatically more potent cytotoxicity compared to progressors (medians 66.95% versus 8.1%, respectively, P<0.001, FIG. 3B). Cytotoxicity was remarkably rapid and complete with day 6 HIV-specific CD8+ T-cells of LTNP frequently killing >70% of targets during the 1-hour incubation. Examination of the outliers suggested an association among proliferation, upregulation of effector molecules and cytotoxicity; i.e., cells from the one LTNP with a low cytotoxic response had undergone less peptide-induced expansion, and the cells of the 3 HLA B*27/57+ progressors (patients 21, 132 and 144) with relatively high cytotoxic responses had exhibited greater proliferation following 6-day peptide stimulation. Cytotoxic capacity on a per-cell basis was similar using day 0 cells from LTNP or progressors over the shared range of E:T ratios (P=0.27, FIG. 3C). In contrast, using day 6 cells, the cytotoxic response curves were significantly different between LTNP and progressors (P=0.03, FIG. 3D). The potent ability of LTNP CD8+ T-cells to lyse target cells was highly efficient and observed down to E:T ratios as low as 2-3:1.
HIV-Specific CD8+ T-Cells from LTNP Mediate Potent Cytotoxicity of HIV-Infected Primary Autologous CD4+ T-Cell Targets

A clear determination whether the diminished cytotoxic responses of progressors relative to LTNP were due to lower CD8+ T-cell numbers or reduced per-cell cytotoxic capacity was difficult to establish at the low E:T ratios observed with peptide-pulsed targets. Therefore, a system to measure CD8+ T-cell-mediated cytotoxicity of autologous, acutely HW-infected CD4+ T-cells was developed (Sacha et al., 2007), permitting a sampling of a broader array of cells specific for other HIV-encoded peptides and thereby, the total cytotoxic responses at higher E:T ratios. In addition, a LIVE/DEAD reagent that enabled separation of 3 cell populations: effectors, targets, and targets that were dead prior to the 1-hour co-incubation was used (FIG. 4A). In this assay, CD8+ effector frequency (based upon IFN-gamma secretion) and target cell GrB activity were measured in parallel (FIGS. 4B and 4C). Following analysis for GrB activity, the fraction of targets expressing HIV p24 was quantified in the same samples by flow cytometry, and the infected CD4+ T-cell elimination (ICE) was determined as another measure of cytotoxic T-cell efficacy (FIG. 4D). The association between GrB target cell activity and cell death in this system was verified by the observation of increased membrane permeability to propidium iodide (PI) only in the infected target cells exhibiting increased GrB activity (FIG. 8A). Furthermore, the responses measured by GrB activity or ICE were abrogated when CD8+ T-cells were incubated with autologous un-infected or heterologous, HLA-mismatched infected targets, confirming that cytotoxicity was mediated by HIV-specific CD8+ T-cells in an HLA-restricted fashion (FIG. 8B). In summary, this combination of techniques then permitted accurate measurements of HIV-specific CD8+ T-cell frequency, delivery of functional granzyme B into infected lymphoblast targets, and infected target cell frequency and elimination (FIGS. 4A-D).

Using day 0 CD8+ T-cells, the cytotoxic responses measured by GrB activity or ICE were comparable between patient groups, except for significant differences in ICE between LTNP and Rx<50 (P<0.001, FIGS. 5A, 5B, left panels). Although cytotoxicity measured by either method was significantly greater using day 6 cells compared with day 0 cells for each patient group, day 6 CD8+ T-cells derived from LTNP had markedly greater cytotoxic capacity than either progressors or Rx<50 (P<0.001 for all comparisons, FIGS. 5A, 5B, right panels). A very strong correlation was noted between GrB target cell activity and ICE when day 6 cells were used (R=0.79, P<0.001; FIG. 5C). In a subset of 18 patients, perforin content was tightly correlated with GrB target cell activity and ICE (R=0.9, P<0.001- and R=0.8,.P<0.001, respectively; FIGS. 5D, 5E). These results suggest that cytotoxicity measured by either method is highly dependent upon memory cell lytic granule loading.

The data were also analyzed on a per-cell basis using measured E:T ratios. Using day 0 cells, target cell GrB activity was not significantly different between LTNP and progressors (P>0.5, FIG. 6A, top panel). ICE was modestly but significantly greater in LTNP than in progressors over the common range of E:T ratios (P=0.03, FIG. 6B, top panel). In contrast to the results with day 0 cells, GrB activity and ICE mediated by day 6 cells were significantly greater for LTNP than progressors by a constant 18% and 40%, respectively, over the common range of E:T ratios (P<0.001, FIGS. 6A, 6B, bottom panels). The minimal overlap of E:T ratios between Rx<50 and the other groups precluded meaningful comparisons. Diminished per-cell cytotoxicity in progressors compared to LTNP was not due to death of HIV-specific CD8+ T-cells given the persistence of high frequencies of IFN-gamma+ cells 6 hours later. Although progressor-derived cells were able to activate and produce TN-gamma, cytotoxicity mediated by these cells measured either by GrB activity or ICE, never reached the levels observed in LTNP, even at high E:T ratios. The differences in cytotoxic responses between LTNP and other patient groups were consistent with, and more dramatic than, those measured against peptide-pulsed targets. These data suggest the cytotoxic capacity of HIV-specific CD8+ T-cells of LTNP is attributable not merely to increases in cell numbers, but also to qualitative changes in effector cells. Furthermore, the observation that the cytotoxic capacities of these cells are not significantly restored in patients with suppressed viremia due to ART supports that diminished cytotoxicity of untreated progressors' cells is not simply a consequence of high levels of antigen. These results suggest that elimination of autologous HIV-infected CD4+ T-cells mediated by the granule exocytosis pathway segregates with immunologic control of HIV. They also demonstrate that lytic granule contents of memory cells are an important determinant of cytotoxicity that must be induced for maximal per-cell killing capacity.

### Diminished Cytotoxic Capacity of HIV-Specific CD8+ T-Cells of Progressors is Reversible

Several features of HIV-specific CD8+ T-cells of progressors are consistent with some states of anergy, including diminished proliferative capacity and IL-2 production (Betts et al., 2006; Migueles et al., 2002; Zimmerli et al., 2005). A number of stimuli have been reported to overcome the anergic state (reviewed in (Schwartz, 2003)). Stimulation with phorbol-12-myristate-13-acetate and ionomycin (D24-PMA/Io), followed by a period of rest prior to re-stimulation with HIV peptides and IL-2, produced frequencies of HIV-specific CD8+ T-cells that were greater than those produced by treatment with anti-CD3/CD28, a period of rest, and re-stimulation with HW peptides and IL-2 (D24-CD3/28, P=0.03; FIGS. 7A, 7B, 9-11). To analyze whether these frequencies translated into changes in cytotoxic capacity, a regression analysis of GrB activity on the log of the E:T ratio was performed for the three sets of conditions (D24-PMA/Io, D24-CD3/28 and cells stimulated for 6 days with Gag peptides (D6-Gag), FIGS. 7C-7E). For a fixed E:T ratio of 1, there was a statistically significant difference for D24-PMA/Io versus D24-CD3/28 (51.4 versus 28.6%, P<0.001) and for D24-PMA/Io versus D6-Gag (51.4 versus 19.8%, P<0.05), but not for D24-CD3/28 versus D6-Gag (P>0.5). Therefore, unlike anti-CD3/anti-CD28 and IL-2-treated cells, PMA/Io-treated cells mediated significantly greater cytotoxicity of peptide-pulsed targets compared with D6 Gag cells in a manner that overlapped with activity of cells from LTNP (FIGS. 7C-7E).

The recovery of proliferation of HIV-specific CD8+ T-cells of progressors by PMA/Io and prior descriptions of diminished IL-2 production by these cells (Betts et al., 2006; Zimmerli et al., 2005) suggested that there may be some disruption of the calcineurin-nuclear factor of activated T-cells (NFAT) pathway. Nuclear translocation of NFAT is an important early signal leading to cell division and IL-2 transcription (reviewed in (Sundrud and Rao, 2007)). This pathway was examined using a technique that allows for quantitative image analysis and flow cytometry in a single platform. Greater NFAT nuclear translocation in the HIV-specific cells of LTNP (median 77.16%) than those of untreated (52.34%; p=0.05) or treated progressors was observed (26.08%; p=0.002, FIG. 7F). Although only a limited number of specificities can be examined by this technique and there is some overlap between patient groups, these data suggest that a greater fraction of HIV-specific cells of LTNP maintain the ability to translocate NFAT to the nucleus upon antigen encounter.

The use of phorbol 12-myristate 13-acetate (PMA) and ionomycin (Io) can “release the brake” that was preventing progressors' CD8+ T cells from proceeding through cell cycle. In initial experiments wherein PMA/Io was added directly to cultures, most of the plated cells died, presumably as a result of overwhelming activation-induced cell death. Viability was significantly improved when the cells were stimulated for 6 hours with PMA/Io or anti-CD3/anti-CD28 monoclonal antibodies, washed and then plated for some time. As shown in FIG. 9, a time course was performed to identify the conditions leading to greatest proliferation, and, therefore, the highest numbers, of viable HIV-specific CD8+ T cells. It was found that a 6-hour stimulation with PMA 6.5 nM and Io 0.2 μM followed by some washes to remove any residual PMA/Io and an 18-day (versus a 6- or 12-day) period of rest before re-stimulating these cells with 2 IU/ml of IL-2 and HIV antigens (pools of 15-mer peptides, each peptide at a final concentration of 2 μg/ml) for 6 more days (24-day total culture) induced the greatest proliferation of HIV-specific CD8+ T cells compared with other polyclonal stimuli (e.g., anti-CD3/anti-CD28, each at 1 μg/ml; FIGS. 9, 10). As seen in FIG. 10, the highest frequencies of HIV-specific CD8+ T cells were obtained under these conditions (black bars in lower row and “day 24” column). The final design that appeared to yield the greatest proliferation and highest frequencies of HIV-specific CD8+ T cells is summarized in FIG. 11. During the 18-day rest, top medium was replaced every 6 days with fresh medium.

The greater proliferation of cells resulting from stimulation with PMA/Io compared with anti-CD3/anti-CD28 was associated with significantly greater killing (FIGS. 7A-E). Furthermore, these increases in killing carried out by PMA/Io-treated CD8+ T cells in progressors were comparable to the results observed using LTNP CD8+ T cells not stimulated with PMA/Io. In other words, defective proliferation was restored, which was also associated with improved killing capacity. Thus, potent polyclonal stimulation with PMA/Io, a period of rest and re-stimulation with HIV antigens in the presence of IL-2 in vitro can induce the cells of progressors to proceed through cell cycle and to undergo all of the listed downstream effects culminating in the elimination of HIV-infected CD4+ T cells in a manner that is strikingly similar to results observed with LTNP cells

Throughout this application, various publications are referenced. The disclosures of these publications in their entireties are hereby incorporated by reference into this application in order to more fully describe the state of the art to which this invention pertains.

### REFERENCES

Adnan, S., Balamurugan, A., Trocha, A., Bennett, M. S., Ng, H. L., Ali, A., Brander, C., and Yang, O. O. (2006). Nef interference with HIV-1-specific CTL antiviral activity is epitope specific. Blood 108, 3414-3419.
Appay, V., Dunbar, P. R., Callan, M., Klenerman, P., Gillespie, G. M., Papagno, L., Ogg, G. S., King, A., Lechner, F., Spina, C. A., et al. (2002). Memory CD8+ T cells vary in differentiation phenotype in different persistent virus infections. Nat Med 8, 379-385.
Arrode, G., Finke, J. S., Zebroski, H., Siegal, F. P., and Steinman, R. M. (2005). CD8+ T cells from most HIV-1-infected patients, even when challenged with mature dendritic cells, lack functional recall memory to HIV gag but not other viruses. European journal of immunology 35, 159-170.
Bailey, J. R., Williams, T. M., Siliciano, R. F., and Blankson, J. N. (2006). Maintenance of viral suppression in HIV-1-infected HLA-B*57+ elite suppressors despite CTL escape mutations. J Exp Med 203, 1357-1369.
Barber, D. L., Wherry, E. J., and Ahmed, R. (2003). Cutting edge: rapid in vivo killing by memory CD8 T cells. J Immunol 171, 27-31.
Betts, M. R., Ambrozak, D. R., Douek, D. C., Bonhoeffer, S., Brenchley, J. M., Casazza, J. P., Koup, R. A., and Picker, L. J. (2001). Analysis of total human immunodeficiency virus (HIV)-specific CD4(+) and CD8(+) T-cell responses: relationship to viral load in untreated HIV infection. J Virol 75, 11983-11991.
Betts, M. R., Brenchley, J. M., Price, D. A., De Rosa, S. C., Douek, D. C., Roederer, M., and Koup, R. A. (2003). Sensitive and viable identification of antigen-specific CD8+ T cells by a flow cytometric assay for degranulation. J Immunol Methods 281, 65-78.
Betts, M. R., Nason, M. C., West, S. M., De Rosa, S. C., Migueles, S. A., Abraham, J., Lederman, M. M., Benito, J. M., Goepfert, P. A., Connors, M., et al. (2006). HIV nonprogressors preferentially maintain highly functional HW-specific CD8+ T cells. Blood 107, 4781-4789.
Cao, Y., Qin, U., Zhang, L., Safrit, J., and Ho, D. D. (1996). Characterization of long-term survivors of human immunodeficiency virus type 1 infection. Immunol Lett 51, 7-13.
Casazza, J. P., Betts, M. R., Picker, L. J., and Koup, R. A. (2001). Decay kinetics of human immunodeficiency virus-specific CD8+ T cells in peripheral blood after initiation of highly active antiretroviral therapy. J Virol 75, 6508-6516.
Collins, K. L., Chen, B. K., Kalams, S. A., Walker, B. D., and Baltimore, D. (1998). HIV-1 Nef protein protects infected primary cells against killing by cytotoxic T lymphocytes. Nature 391, 397-401.
Davey, R. T., Jr., Bhat, N., Yoder, C., Chun, T. W., Metcalf, J. A., Dewar, R., Natarajan, V., Lempicki, R. A., Adelsberger, J. W., Miller, K. D., et al. (1999). HW-1 and T cell dynamics after interruption of highly active antiretroviral therapy (HAART) in patients with a history of sustained viral suppression. Proc Natl Acad Sci USA 96, 15109-15114.
Emu, B., Sinclair, E., Favre, D., Moretto, W. J., Hsue, P., Hoh, R., Martin, J. N., Nixon, D. F., McCune, J. M., and Deeks, S. G. (2005). Phenotypic, functional, and kinetic parameters associated with apparent T-cell control of human immunodeficiency virus replication in individuals with and without antiretroviral treatment. J Virol 79, 14169-14178.
Flores-Villanueva, P. O., Yunis, E. J., Delgado, J. C., Vittinghoff, E., Buchbinder, S., Leung, J. Y., Uglialoro, A. M., Clavijo, O. P., Rosenberg, E. S., Kalams, S. A., et al. (2001). Control of HIV-1 viremia and protection from AIDS are associated with HLA-Bw4 homozygosity. Proc Natl Acad Sci USA 98, 5140-5145.
Gea-Banacloche, J. C., Migueles, S. A., Martino, L., Shupert, W. L., McNeil, A. C., Ehler, L., Prussin, C., Stevens, R., L., L., Altman, J., et al. (2000). Maintenance of large numbers of virus specific CD8+ T cells in HIV infected progressors and long term nonprogressors. J. Immunol. 165, 1082-1092.
George, T. C., Fanning, S. L., Fitzgeral-Bocarsly, P., Medeiros, R. B., Highfill, S., Shimizu, Y., Hall, B. E., Frost, K., Basiji, D., Ortyn, W. E., et al. (2006). Quantitative measurement of nuclear translocation events using similarity analysis of multispectral cellular images obtained in flow. J Immunol Methods 311, 117-129.
Gray, C. M., Lawrence, J., Schapiro, J. M., Altman, J. D., Winters, M. A., Crompton, M., Loi, M., Kundu, S. K., Davis, M. M., and Merigan, T. C. (1999). Frequency of Class I HLA-Restricted Anti-HIV CD8+ T Cells in Individuals Receiving Highly Active Antiretroviral Therapy (HAART). J Immunol 162, 1780-1788.
Harrer, T., Harrer, E., Kalams, S. A., Barbosa, P., Trocha, A., Johnson, R. P., Elbeik, T., Feinberg, M. B., Buchbinder, S. P., and Walker, B. D. (1996). Cytotoxic T lymphocytes in asymptomatic long-term nonprogressing HIV-1 infection. Breadth and specificity of the response and relation to in vivo viral quasispecies in a person with prolonged infection and low viral load. J Immunol 156, 2616-2623.
Horton, H., Frank, I., Baydo, R., Jalbert, E., Penn, J., Wilson, S., McNevin, J. P., McSweyn, M. D., Lee, D., Huang, Y., et al. (2006). Preservation of T cell proliferation restricted by protective HLA alleles is critical for immune control of HIV-1 infection. J Immunol 177, 7406-7415.
Kaech, S. M., Hemby, S., Kersh, E., and Ahmed, R. (2002). Molecular and functional profiling of memory CD8 T cell differentiation. Cell 111, 837-851.
Kalams, S. A., Goulder, P. J., Shea, A. K., Jones, N. G., Trocha, A. K., Ogg, G. S., and Walker, B. D. (1999). Levels of human immunodeficiency virus type 1-specific cytotoxic T-lymphocyte effector and memory responses decline after suppression of viremia with highly active antiretroviral therapy. J Virol 73, 6721-6728.
Kaslow, R. A., Carrington, M., Apple, R., Park, L., Munoz, A., Saah, A. J., Goedert, J. J., Winkler, C., O'Brien, S. J., Rinaldo, C., et al. (1996). Influence of combinations of human major histocompatibility complex genes on the course of HIV-1 infection. Nat Med 2, 405-411.
Klein, M. R., van Baalen, C. A., Holwerda, A. M., Kerkhof Garde, S. R., Bende, R. J., Keet, I. P., Eeftinck-Schattenkerk, J. K., Osterhaus, A. D., Schuitemaker, H., and Miedema, F. (1995). Kinetics of Gag-specific cytotoxic T lymphocyte responses during the clinical course of HIV-1 infection: a longitudinal analysis of rapid progressors and long-term asymptomatics. J Exp Med 181, 1365-1372.
Lichterfeld, M., Kaufmann, D. E., Yu, X. G., Mui, S. K., Addo, M. M., Johnston, M. N., Cohen, D., Robbins, G. K., Pae, E., Alter, G., et al. (2004). Loss of HIV-1-specific CD8+ T cell proliferation after acute HIV-1 infection and restoration by vaccine-induced HIV-1-specific CD4+ T cells. J Exp Med 200, 701-712.
Lopez Bernaldo de Quiros, J. C., Shupert, W. L., McNeil, A. C., Gea-Banacloche, J. C., Flanigan, M., Savage, A., Martino, L., Weiskopf, E. E., Immamichi, H., Zhang, Y. M., et al. (2000). Resistance to Replication of HIV Challenge Virus in SCID-Hu Mice Engrafted with PBMC of Nonprogressors is Mediated by CD8+ T cells and Associated with a Proliferative Response to p24 Antigen. Journal of Virology 74, 2023-2028.
Meng, Y., Harlin, H., O'Keefe, J. P., and Gajewski, T. F. (2006). Induction of cytotoxic granules in human memory CD8+ T cell subsets requires cell cycle progression. J Immunol 177, 1981-1987.
Migueles, S. A., and Connors, M. (2001). Frequency and function of HIV-specific CD8(+) T Cells. Immunol Lett 79, 141-150.
Migueles, S. A., Laborico, A. C., Imamichi, H., Shupert, W. L., Royce, C., McLaughlin, M., Ehler, L., Metcalf, J., Liu, S., Hallahan, C. W., and Connors, M. (2003). The differential ability of HA B*5701+ long-term nonprogressors and progressors to restrict human immunodeficiency virus replication is not caused by loss of recognition of autologous viral gag sequences. J Virol 77, 6889-6898.
Migueles, S. A., Laborico, A. C., Shupert, W. L., Sabbaghian; M. S., Rabin, R., Hallahan, C. W., Van Baarle, D., Kostense, S., Miedema, F., McLaughlin, M., et al. (2002). HIV-specific CD8+ T cell proliferation is coupled to perforin expression and is maintained in nonprogressors. Nat Immunol 3, 1061-1068.
Migueles, S. A., Sabbaghian, M. S., Shupert, W. L., Bettinotti, M. P., Marincola, F. M., Martino, L., Hallahan, C. W., Selig, S. M., Schwartz, D., Sullivan, J., and Connors, M. (2000). HLA B*5701 is highly associated with restriction of virus replication in a subgroup of HIV-infected long term nonprogressors. Proc Natl Acad Sci USA 97, 2709-2714.
Migueles, S. A., Tilton, J. C., and Connors, M. (2004). Advances in understanding immunologic control of HIV infection. Curr HIV/AIDS Rep 1, 12-17.
Murali-Krishna, K., Lau, L. L., Sambhara, S., Lemonnier, F., Altman, J., and Ahmed, R. (1999). Persistence of memory CD8 T cells in MHC class I-deficient mice. Science 286, 1377-1381.
Navis, M., Schellens, I., van Baarle, D., Borghans, J., van Swieten, P., Miedema, F., Kootstra, N., and Schuitemaker, H. (2007). Viral replication capacity as a correlate of HLA B57/B5801-associated nonprogressive HIV-1 infection. J Immunol 179, 3133-3143.
Ogg, G. S., Jin, X., Bonhoeffer, S., Moss, P., Nowak, M. A., Monard, S., Segal, J. P., Cao, Y., Rowland-Jones, S. L., Hurley, A., et al. (1999a). Decay kinetics of human immunodeficiency virus-specific effector cytotoxic T lymphocytes after combination antiretroviral therapy. J Virol 73, 797-800.
Packard, B. Z., Telford, W. G., Komoriya, A., and Henkart, P. A. (2007). Granzyme B activity in target cells detects attack by cytotoxic lymphocytes. J Immunol 179, 3812-3820.
Palmer, S., Wiegand, A. P., Maldarelli, F., Bazmi, H., Mican, J. M., Polis, M., Dewar, R. L., Planta, A., Liu, S., Metcalf, J. A., et al. (2003). New real-time reverse transcriptase-initiated PCR assay with single-copy sensitivity for human immunodeficiency virus type 1 RNA in plasma. J Clin Microbiol 41, 4531-4536.
Pantaleo, G., Menzo, S., Vaccarezza, M., Graziosi, C., Cohen, O. J., Demarest, Montefiori, D., Orenstein, J. M., Fox, C., Schrager, L. J., et al. (1995). Studies in subjects with long-term nonprogressive human immunodeficiency virus infection. N Engl J Med 332, 209-216.
Rehr, M., Cahenzli, J., Haas, A., Price, D. A., Gostick, E., Huber, M., Karrer, U., and Oxenius, A. (2008). Emergence of polyfunctional CD8+ T cells after prolonged suppression of HW replication by antiretroviral therapy. J Virol.
Sacha, J. B., Chung, C., Rakasz, E. G., Spencer, S. P., Jonas, A. K., Bean, A. T., Lee, W., Burwitz, B. J., Stephany, J. J., Loffredo, J. T., et al. (2007). Gag-specific CD8+ T lymphocytes recognize infected cells before AIDS-virus integration and viral protein expression. J Immunol 178, 2746-2754.
Saez-Cirion, A., Lacabaratz, C., Lambotte, O., Vermisse, P., Urrutia, A., Boufassa, F., Barre-Sinoussi, F., Delfraissy, J. -F., Sinet, M., Pancino, G., and Venet, A. (2007). HIV controllers exhibit potent CD8 T cell capacity to suppress HIV infection ex vivo and peculiar cytotoxic T lymphocyte activation phenotype. Proc Natl Acad Sci USA 104, 6776-6781.
Sandberg, J. K., Fast, N. M., and Nixon, D. F. (2001). Functional heterogeneity of cytokines and cytolytic effector molecules in human CD8+ T lymphocytes. J Immunol 167, 181-187.
Schwartz, R. H. (2003). T cell anergy. Annu Rev Immunol 21, 305-334.
Seder, R. A., and Ahmed, R. (2003). Similarities and differences in CD4+ and CD8+ effector and memory T cell generation. Nat Immunol 4, 835-842.
Shin, H., Blackburn, S. D., Blattman, J. N., and Wherry, E. J. (2007). Viral antigen and extensive division maintain virus-specific CD8 T cells during chronic infection: J Exp Med 204, 941-949.
Shlobin, O. A., West, E. E., Lechtzin, N., Miller, S. M., Borja, M., Orens, J. B., Dropulic, L. K., and McDyer, J. F. (2006). Persistent cytomegalovirus-specific memory responses in the lung allograft and blood following primary infection in lung transplant recipients. J Immunol 176, 2625-2634.
Sundrud, M. S., and Rao, A. (2007). New twists of T cell fate: control of T cell activation and tolerance by TGF-beta and NFAT. Current opinion in immunology 19, 287-293.
Sylwester, A. W., Mitchell, B. L., Edgar, J. B., Taormina, C., Pelte, C., Ruchti, F., Sleath, P. R., Grabstein, K. H., Hosken, N. A., Kern, F., et al. (2005). Broadly targeted human cytomegalovirus-specific CD4+ and CD8+ T cells dominate the memory compartments of exposed subjects. J Exp Med 202, 673-685.
Tilton, J. C., Luskin, M. R., Johnson, A. J., Manion, M., Hallahan, C. W., Metcalf, J. A., McLaughlin, M., Davey, R. T., Jr., and Connors, M. (2007). Changes in paracrine interleukin-2 requirement, CCR7 expression, frequency, and cytokine secretion of human immunodeficiency virus-specific CD4+ T cells are a consequence of antigen load. J Virol 81, 2713-2725.
Trambas, C. M., and Griffiths, G. M. (2003). Delivering the kiss of death. Nat Immunol 4, 399-403.
Wherry, E. J., Barber, D. L., Kaech, S. M., Blattman, J. N., and Ahmed, R. (2004). Antigen-independent memory CD8 T cells do not develop during chronic viral infection. Proc Natl Acad Sci USA 101, 16004-16009.
Wolint, P., Betts, M. R., Koup, R. A., and Oxenius, A. (2004). Immediate cytotoxicity but not degranulation distinguishes effector and memory subsets of CD8+ T cells. J Exp Med 199, 925-936.
Zhang, D., Shankar, P., Xu, Z., Harnisch, B., Chen, G., Lange, C., Lee, S. J., Valdez, H., Lederman, M. M., and Lieberman, J. (2003). Most antiviral CD8 T cells during chronic viral infection do not express high levels of perforin and are not directly cytotoxic. Blood 101, 226-235.
Zimmerli, S. C., Harari, A., Cellerai, C., Vallelian, F., Bart, P. A., and Pantaleo, G. (2005). HIV-1-specific IFN-gamma/M-2-secreting CD8 T cells support CD4-independent proliferation of HW-1-specific CD8 T cells. Proc Natl Acad Sci USA 102, 7239-7244.

TABLE 1
Characteristics of HIV-Infected Long-Term Nonprogressors

| | | CD4 T Cell | CD8 T Cell | | HLA | | | |
|---|---|---|---|---|---|---|---|---|
| Patient | Diagnosis | Count | Count | HIV-1 RNA | Class I | | | SCA* |
| Number | Year | Cells/mL | Cells/mL | Copies/mL | A | B | C | Copies/mL |
| 4 | 1985 | 1,063 | 1,088 | <50 | 1.31 | 8.57 | 6.7 | 8 |
| 5 | 1985 | 1,105 | 835 | <50 | 2.24 | 57 | 6.7 | 2 |
| 6 | 1986 | 760 | 803 | <50-62 | 11.30 | 52.57 | 7.12 | 1 |
| 7 | 1985 | 277 | 385 | <50 | 1.2 | 57 | 6 | 4 |
| 8 | 1985 | 664 | 1,120 | <50-930 | 11.23 | 44.57 | 4.6 | |
| 9 | 1997 | 1,079 | 985 | <50 | 23.26 | 44.57 | 1.7 | 4 |
| 10 | 1996 | 602 | 584 | <50 | 1.33 | 50.57 | 6 | <1 |
| 12 | 1986 | 500 | 218 | <50 | 3.11 | 7.57 | 6.7 | 28 |
| 13 | 1986 | 1,016 | 767 | <50 | 1.11 | 35.57 | 4.6 | <1 |
| 25 | 1986 | 1,028 | 1,082 | <50-1,089 | 3.24 | 27.57 | 2.6 | |
| 30 | 1990 | 422 | 592 | <50 | 31.74 | 51.57 | 7.16 | 2 |
| 33 | 1995 | 955 | 881 | <50 | 2.30 | 13.57 | 6 | <1 |
| 34 | 1989 | 1,746 | 1,164 | <50 | 1.2 | 8.57 | 6.7 | 48 |
| 37 | 1998 | 1,616 | 707 | <50 | 30 | 42.57 | 17.18 | 16.3 |
| 38 | 1990 | 1,329 | 1,243 | <50 | 2.24 | 44.57 | 5.6 | 1 |
| 47 | 1994 | 500 | 1,284 | <50-137 | 1.74 | 57.81 | 7.18 | 4 |
| 58 | 1989 | 485 | 277 | <50 | 30.74 | 15.57 | 3.8 | <1 |
| 59 | 1986 | 833 | 549 | <50-125 | 23.30 | 7.57 | 7.15 | |
| 65 | 1993 | 865 | 388 | <50 | 30.74 | 14.57 | 2.8 | 4 |
| 66 | 1992 | 1,488 | 713 | <50 | 2.30 | 13.57 | 6 | <1 |
| 68 | 1986 | 1,362 | 1,055 | <50 | 3.29 | 57.81 | 18 | 5 |
| 71 | 1996 | 1,300 | 816 | <50 | 1.24 | 38.57 | 6.12 | <1 |
| 73 | 1991 | 801 | 1,012 | <50 | 2.3 | 7.57 | 6.7 | 1 |
| 75 | 1987 | 492 | 505 | <50-304 | 1.2 | 37.57 | 6 | <1 |
| 77 | 1999 | 513 | 1,441 | <50-142 | 33.74 | 53.57 | 4.18 | |
| 79 | 1998 | 520 | 780 | <50 | 1.30 | 42.57 | 7.17 | <1 |
| 81 | 2001 | 780 | 739 | <50 | 1.29 | 52.57 | 6.12 | |
| 3 | 1985 | 915 | 1,079 | <50 | 2.3 | 13.39 | 6.7 | |
| 17 | 1985 | 1,073 | 1,051 | <50 | 2.26 | 27.38 | 1.12 | 1.5 |
| 32 | 1994 | 785 | 328 | <50 | 1.32 | 8.27 | 1.7 | 8.3 |
| 48 | 1989 | 834 | 417 | <50 | 1.33 | 8.53 | 1.4 | 19.3 |
| 49 | 1992 | 1,084 | 784 | <50 | 3.68 | 40.53 | 2.4 | 23 |
| 53 | 1992 | 880 | 448 | <50 | 11.80 | 27.35 | 2.4 | 32 |
| 60 | 1985 | 992 | 1,110 | <50 | 3.11 | 35.51 | 4.15 | 9.7 |
| 61 | 2004 | 751 | 594 | <50 | 2.29 | 44.49 | 7.16 | 1.7 |
| 62 | 1985 | 1,452 | 807 | <50 | 1.32 | 35.73 | 4.15 | <1 |
| 67 | 1982 | 1,308 | 880 | <50-201 | 32 | 27.44 | 1.5 | 43.4 |
| 72 | 1998 | 1,813 | 594 | <50 | 30.33 | 42.58 | 2.17 | <1 |
| 74 | 1988 | 851 | 890 | <50 | 11.32 | 35.50 | 4.6 | <1 |
| 76 | 2001 | 861 | 698 | <50 | 23 | 58.81 | 7.18 | 2.6 |
| 80 | 2003 | 520 | 328 | <50 | 2.31 | 44.51 | 5.15 | <1 |

*Single copy assay

TABLE 2
Characteristics of HIV-Infected Progressors

| Patient | Diagnosis | CD4 T Cell | CD8 T Cell | HIV-1 RNA | HLA Class I | | | SCA*^{†} |
|---|---|---|---|---|---|---|---|---|
| Number | Year | Count Cells/mL | Count Cells/mL | Copies/mL | A | B | C | Copies/mL |

Slow P^{§}

| | | | | | | | |
|---|---|---|---|---|---|---|---|
| 20 | 1985 | 1040 | 894 | 28890 | 1 | 52.57 | 6.12 |
| 21 | 1984 | 721 | 811 | 10930 | 1.2 | 8.27 | 1.7 |
| 44 | 1986 | 826 | 1386 | 4750 | 1.24 | 27.37 | 2.6 |
| 45 | 1990 | 647 | 1671 | 12709 | 2 | 45.57 | 6 |
| 46 | 1984 | 557 | 525 | 3237 | 2.23 | 53.57 | 6.18 |
| 50 | 2001 | 752 | 1444 | 15129 | 2.11 | 8.51 | 4.7 |
| 63 | 1986 | 803 | 1109 | 4137 | 30 | 57.81 | 18 |

Viremic P^{§}

| | | | | | | | |
|---|---|---|---|---|---|---|---|
| 107 | 1987 | 445 | 1674 | 120291 | 3 | 40.57 | 3.7 |
| 131 | 1989 | 238 | 1017 | 85981 | 2.11 | 35.57 | 4.6 |
| 133 | 2000 | 331 | 1513 | 35343 | 33.6 | 15.35 | 3.16 |
| 134 | 2003 | 274 | 325 | 45569 | 68 | 15.27 | 3 |
| 138 | 1996 | 444 | 2331 | 160954 | 1.68 | 15.57 | 1.7 |
| 139 | 1993 | 453 | 861 | 78984 | 2.32 | 27.35 | 1.4 |
| 142 | 1994 | 387 | 944 | 175204 | 2.68 | 27.40 | 1.3 |
| 143 | 1985 | 323 | 707 | 60577 | 2.11 | 15.27 | 1.4 |
| 148 | 1999 | 243 | 757 | 94919 | 2.3 | 27.42 | 2.17 |
| 149 | 1991 | 739 | 979 | 30733 | 3.24 | 7 | 7.15 |
| 150 | 2001 | 790 | 1867 | 144497 | 30 | 57.7 | 15.1 |
| 203 | 1989 | 572 | 1122 | 48876 | 30.3 | 18.57 | 5.6 |

Treated (VL > 1000)

| | | | | | | | |
|---|---|---|---|---|---|---|---|
| 103 | 1991 | 457 | 977 | 5054 | 2.11 | 55.57 | 3.6 |
| 104 | 1988 | 332 | 705 | 1702 | 2 | 57.58 | 3.6 |
| 105 | 1990 | 463 | 990 | 7299 | 2.80 | 8.57 | 7.12 |
| 144 | 1986 | 262 | 665 | 8797 | 3.26 | 7.57 | 6.7 |
| 154 | 1985 | 224 | 881 | 9664 | 1.2 | 27.35 | 2.4 |
| 155 | 2000 | 304 | 1449 | 5429 | 1.23 | 57.81 | 7.8 |

Treated (VL < 50)

| | | | | | | | | |
|---|---|---|---|---|---|---|---|---|
| 27 | 1993 | 466 | 519 | <50 | 2.36 | 15.42 | 3.17 | <1 |
| 101 | 1986 | 632 | 902 | <50 | 1.31 | 51.57 | 6.15 | 2.3 |
| 113 | 1991 | 510 | 1021 | <50 | 1.2 | 45.57 | 7.16 | 1.4 |
| 114 | 1989 | 290 | 391 | <50 | 1.24 | 8.57 | 6.7 | <1 |
| 126 | 1991 | 829 | 1525 | <50 | 1.68 | 8.57 | 7.18 | <1 |
| 127 | 1994 | 720 | 702 | <50 | 3.24 | 7.18 | 7 | 5 |
| 129 | 1988 | 445 | 783 | <50 | 2.32 | 15.27 | 1.2 | 3 |
| 132 | 1995 | 1409 | 479 | <50 | 30.6 | 40.57 | 3.7 | <1 |
| 141 | 2001 | 408 | 553 | <50 | 2.24 | 35.49 | 3.7 | 13.8 |
| 151 | 1994 | 515 | 1245 | <50 | 2.23 | 45.57 | 16.1 | <1 |
| 153 | 1993 | 968 | 1338 | <50 | 2 | 15.57 | 3.6 | 1 |
| 157 | 1987 | 626 | 1065 | <50 | 2.24 | 52.57 | 6.12 | <1 |
| 158 | 2001 | 959 | 750 | <50 | 1.32 | 8.35 | 4.7 | 13 |
| 159 | 1997 | 781 | 521 | <50 | 11 | 15 | 8 | <1 |
| 160 | 1996 | 922 | 820 | <50 | 26.6 | 15.18 | 4.5 | <1 |
| 161 | 2001 | 521 | 695 | <50 | 11.6 | 27 | 2.7 | 1 |
| 162 | 1997 | 659 | 678 | <50 | 3 | 14.35 | 4.8 | <1 |
| 163 | 1987 | 668 | 789 | <50 | 24.3 | 18.49 | 7.12 | 14 |
| 164 | 1987 | 1004 | 938 | <50 | 24.3 | 7.27 | 2.7 | 5 |
| 165 | 2001 | 604 | 935 | <50 | 11.2 | 35 | 4 | 2 |
| 166 | 1994 | 247 | 447 | <50 | 2.11 | 8.15 | 3.8 | 4 |
| 167 | 1986 | 649 | 508 | <50 | 2.3 | 14.15 | 3.8 | <1 |
| 168 | 1990 | 1167 | 1228 | <50 | 3.32 | 14 | 8 | <1 |
| 169 | 1989 | 476 | 987 | <50 | 2.3 | 7.14 | 7.8 | <1 |
| 170 | 1994 | 561 | 546 | <50 | 23.6 | 7.53 | 4.7 | 29 |
| 171 | 1995 | 589 | 828 | <50 | 25.2 | 8.49 | 7 | 1 |
| 172 | 1988 | 729 | 1200 | <50 | 2 | 13.35 | 3 | <1 |
| 173 | 1988 | 599 | 889 | <50 | 2 | 15.58 | 3 | <1 |
| 174 | 1992 | 675 | 459 | <50 | 33.7 | 42.53 | 4.17 | <1 |
| 175 | 1994 | 532 | 627 | <50 | 11.2 | 35.40 | 3.4 | <1 |
| 176 | 2000 | 382 | 665 | <50 | 2.23 | 45.58 | 6 | 1 |
| 177 | 1985 | 555 | 750 | <50 | 1.2 | 7.40 | 3.7 | 4 |
| 178 | 2002 | 498 | 227 | <50 | 2.30 | 45.50 | 4.16 | <1 |
| 179 | 1990 | 501 | 866 | <50 | 2.25 | 7.44 | 5.7 | 10 |
| 185 | 1991 | 512 | 668 | <50 | 34.3 | 7.44 | 7 | 4 |
| 186 | 1986 | 383 | 544 | <50 | 2.11 | 51.55 | 2.3 | 19 |
| 187 | 1998 | 307 | 247 | <50 | 2.30 | 18.58 | 5.6 | <1 |
| 188 | 1990 | 642 | 708 | <50 | 1.24 | 7.8 | 7 | 2.6 |
| 189 | 1986 | 800 | 990 | <50 | | | | 1 |
| 190 | 1995 | 729 | 496 | <50 | 2.68 | 35.44 | 4.5 | <1 |
| 191 | 2004 | 562 | 1002 | <50 | 29 | 13.51 | 1.6 | <1 |
| 192 | 1997 | 568 | 1237 | <50 | 2.74 | 7.58 | 3.7 | 53 |

*Single copy assay
^{§}Progressors
^{†}P = 0.33 for comparison of SCA results between LTNP (n = 35) and Treated P VL < 50 (n = 42).

TABLE 3
Human Leukocyte Antigen Class I Tetramers

| | | |
|---|---|---|
| Tetramer | HIV-1 | |
| Name* | Protein | Amino Acid Sequence |

| | | | |
|---|---|---|---|
| A1 GY9 | Gag p17 | GSEELRSLY | 71-79 |
| A2 IV9 | RT | ILKEPVHGV | 309-317 |
| A2 SL9 | Gag p17 | SLYNTVATL | 77-85 |
| A3 KK9 | Gag p17 | KIRLRPGGK | 18-26 |
| A3 QK10 | Nef | QVPLRPMTYK | 73-82 |
| A3 RK9 | Gag p17 | RLRPGGKKK | 20-28 |
| A24 RF10 | Nef | RYPLTFGWCF | 134-143 |
| B7 FL9 | Nef | FPVTPQVPL | 68-76 |
| B7 IL9 | gp160 | IPRRIRQGL | 843-851 |
| B8 EI8 | Gag p24 | EIYKRWII | 260-267 |
| B8 FL8 | Nef | FLKEKGGL | 90-97 |
| B27 IK9 | Gag p17 | IRLRPGGKK | 19-27 |
| B27 KK10 | Gag p24 | KRWIILGLNK | 263-272 |
| B35 RY11 | Nef | RPQVPLRPMTY | 71-81 |
| B57 IW9 | Gag p24 | ISPRTLNAW | 147-155 |
| B57 KF11 | Gag p24 | KAFSPEVIPMF | 162-172 |
| B57 QW9 | Gag p24 | QASQEVKNW | 308-316 |

*Abbreviated as HLA class I restriction element followed by HIV peptide sequence identified as first and last amino acid symbols followed by sequence length.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising immune cells of a subject with HIV, wherein the immune cells are activated by contact with a phorbol ester and a calcium ionophore.

2. The composition of claim 1, wherein the immune cell is an HIV-specific CD8⁺ T-cell.

3. The composition of claim 2, wherein the CD8⁺ T-cell is contacted in vitro.

4. The composition of claim 1, wherein the phorbol ester is phorbol-12-myristate-13-acetate (PMA).

5. The composition of claim 1, wherein the calcium inophore is ionomycin.

6. The composition of claim 1, wherein the subject is a progressor.

7. A method of activating an immune cell of a subject with HIV, comprising contacting the immune cell with a phorbol ester and a calcium ionophore, whereby contacting the cell with the phorbol ester and the calcium ionophore activates the cell.

8. The method of claim 7, wherein the immune cell is an HIV-specific CD8⁺ T-cell.

9. The method of claim 8, wherein the CD8⁺ T-cell is contacted in vitro.

10. The method of claim 7, wherein the phorbol ester is PMA.

11. The method of claim 7, wherein the calcium ionophore is ionomycin.

12. The method of claim 7, wherein the subject is a progressor.

13. A method of producing an immune response in a cell from a subject with HW directed against an HIV-infected cell, comprising contacting the immune cell with a phorbol ester and a calcium ionophore, whereby contacting the immune cell with the phorbol ester and the calcium ionophore produces an immune response in the cell directed against the HIV-infected cell.

14. The method of claim 13, wherein the immune cell is an HIV-specific CD8⁺ T-cell.

15. The method of claim 14, wherein the CD8⁺ T-cell is contacted in vitro.

16. The method of claim 13, wherein the phorbol ester is PMA.

17. The method of claim 13, wherein the calcium ionophore is ionomycin.

18. The method of claim 13, wherein the subject is a progressor.

19. A method of increasing production of an effector molecule in an immune cell of a subject with HIV, comprising contacting the immune cell with a phorbol ester and a calcium ionophore, whereby contacting the immune cell with the phorbol ester and the calcium ionophore increases production of the effector molecule in the cell.

20. The method of claim 19, wherein the immune cell is an HIV-specific CD8⁺ T-cell.

21. The method of claim 20, wherein the CD8⁺ T-cell is contacted in vitro.

22. The method of claim 19, wherein the phorbol ester is PMA.

23. The method of claim 19, wherein the calcium ionophore is ionomycin.

24. The method of claim 19, wherein the subject is a progressor.

25. The method of claim 19, wherein the effector molecule is granzyme B.

26. The method of claim 19, wherein the effector molecule is perforin.

27. A method of restoring to an immune cell of a subject with HIV the ability to proliferate, comprising contacting the immune cell with a phorbol ester and a calcium ionophore, whereby contacting the cell with the phorbol ester and the calcium ionophore restores to the immune cell the ability to proliferate.

28. The method of claim 27, wherein the immune cell is an HIV-specific CD8⁺ T-cell.

29. The method of claim 28, wherein the CD8⁺ T-cell is contacted in vitro.

30. The method of claim 27, wherein the phorbol ester is PMA.

31. The method of claim 27, wherein the calcium ionophore is ionomycin.

32. The method of claim 27, wherein the subject is a progressor.

33. A method of increasing the cytotoxicity of an HIV-specific CD8⁺ T-cell for CD4⁺ HW-infected cells of a subject, comprising contacting the HIV-specific CD8⁺ T-cell with a phorbol ester and a calcium ionophore, whereby contacting the HIV-specific CD8⁺ T-cell with the phorbol ester and the calcium ionophore increases the cytotoxicity of the HW-specific CD8⁺ T-cell for CD4⁺ HIV-infected cells of the subject.

34. The method of claim 33, wherein the CD8⁺ T-cell is contacted in vitro.

35. The method of claim 33, wherein the phorbol ester is PMA.

36. The method of claim 33, wherein the calcium ionophore is ionomycin.

37. The method of claim 33, wherein the subject is a progressor.

38. A method of producing an immune response to HIV in a subject diagnosed with HIV, comprising administering to the subject a composition comprising immune cells of the subject, wherein the immune cells are activated by contact with a phorbol ester and a calcium ionophore in vitro.

39. The method of claim 38, wherein the immune cell is an HIV-specific CD8⁺ T-cell.

40. The method of claim 38, wherein the phorbol ester is PMA.

41. The method of claim 38, wherein the calcium ionophore is ionomycin.

42. The method of claim 38, wherein the subject is a progressor.
